(19) United States
(12) Patent Application Publication
(10) Pub. No.: US 2024/0217995 A1
(43) Pub. Date: Jul. 4, 2024

(54) COMPOSITIONS FOR CHEMICAL SYNTHESIS OF PEPTIDES

(71) Applicant: Sederma, Le Perray-en-Yvelines (FR)

(72) Inventor: Cole Seifert, Helena, AL (US)

(73) Assignee: Sederma, Le Perray-en-Yvelines (FR)

(21) Appl. No.: 18/287,257

(22) PCT Filed: Apr. 22, 2022
(86) PCT No.: PCT/US2022/071870

§ 371 Date: Oct. 17, 2023

Related U.S. Application Data
(60) Provisional application No. 63/201,313, filed on Apr. 23, 2021.

(51) Int. Cl. C07F 9/36; C07K 1/10

(52) U.S. Cl. CPC C07F 9/36; C07K 1/10

(57) ABSTRACT

The disclosure relates to compositions that can serve as anchors for the chemical synthesis of peptides. Anchor molecules can include GAP constituents, linker constituents, amino acid constituents, and/or stopper constituents. Anchor molecules can also include anchor peptides, wherein an anchor peptide can be removably coupled with an amino acid of a given sequence and act as a GAP anchor by achieving solubility control over the target peptide as it is synthesized via the addition of one or more other amino acids: the anchor peptide can then be removed from the target peptide. A novel method of peptide synthesis that utilizes novel anchor molecules and/or anchor peptides is also presented.

### CROSS-REFERENCE TO RELATED APPLICATIONS

This application claims priority to U.S. Prov. Pat. App. Ser. No. 63/201,313, entitled “Compositions for Chemical Synthesis of Peptides,” filed Apr. 23,2021. This application cross-references: i) PCT Application No. PCT/US16/68112, “System and method for solution phase GAP peptide synthesis,” filed on 21^{st} of Dec., 2016; ii) PCT Application No. PCT/US19/29569, “Method for Solution-Phase Peptide Synthesis,” filed Apr. 29, 2019; iii) PCT Application No. PCT/US19/33296, “Method for Solution-Phase Peptide Synthesis and Protecting Strategies Thereof,” filed on May 21, 2019; iv) PCT Application No. PCT/US20/15132, filed Jan. 26, 2020; v) U.S. application Ser. No. 17/104, 166, filed Nov. 25, 2020 and published as US2021/0079036 A1, entitled “Compositions and Methods for Chemical Synthesis”; and vi) U.S. Prov. Pat. App. Ser. No. 63/201,313, entitled “Compositions for Chemical Synthesis of Peptides,” filed Apr. 23, 2021; and these applications are herein incorporated by reference as examples.

### STATEMENT OF FEDERALLY SPRONSORED RESEARCH OR DEVELOPMENT

None.

### REFERENCE TO A SEQUENCE LISTING, A TABLE, OR A COMPUTER LISTING COMPACT DISC APPENDIX

None.

### BACKGROUND OF THE INVENTION

Recent research efforts have made significant advancements in the area of purification chemistry, focusing specifically on avoiding column chromatography and recrystallization. This research has been defined as Group-Assisted Purification (GAP) chemistry/technology as a chemistry for organic synthesis that avoids traditional purification methods such as chromatography and/or recrystallization by purposefully introducing a well-functionalized group in the starting material or in the newly generated product. These GAP groups can also often be used as protecting groups to prevent undesired side-reactions during the synthesis of target molecules. Such research has the potential to encompass the entire field of synthetic organic chemistry.

Protecting groups are found in almost every complex synthesis where multiple functional groups are present. Effective protecting groups need to be robust to a wide variety of conditions and must be added and removed with high yield. Protecting groups are used extensively in peptide synthesis, either for solid or solution phase approaches. One of the most commonly used protecting group strategies for peptide synthesis is Fmoc/tBu. Fmoc/tBu chemistry is extremely attractive for peptide synthesis for several reasons, such as the availability of starting materials, cost of production, and the comparatively mild conditions needed for deprotection. U.S. Pat. No. 8,383,770 B2 teaches the use of the Fluorenylmethoxycarbonyl (Fmoc) and tert-Butyloxycarbonyl (Boc) N-terminus protecting groups in Solid-Phase Peptide Synthesis (SPPS), and this technology is well known and widely applied in industry.

Developed by Merrifield in the 1960s, SPPS has become a standard protocol used by multiple scientific disciplines for research and manufacturing. The key disadvantage of SPPS lies in the difficulty of scale-up: many polymer supports are expensive and occupy the vast majority of the mass of the material to be worked with. Coupling reactions in SPPS are also inefficient because the reactions occur on a solid-liquid interface. Additionally, after each deprotection and coupling reaction, the resin must be washed with solvent to remove any impurities generated from previous reactions, and this generates significant solvent waste that can be extremely problematic on a large scale.

However, despite these disadvantages of SPPS and the attractiveness of Fmoc/tBu chemistry, examples of economically feasible Fmoc protection schemes for solution-phase peptide synthesis (SolPPS) are rare. U.S. Pat. No. 5,516,891 A provides one of the few examples of Fmoc-based SolPPS. Again, the Fmoc peptide synthesis is almost entirely restricted to SPPS, due to the formation of N-fluorenylmethylpiperidine (NFMP) as a side product during deprotection, which is difficult to remove without polymer supports. The standard protocol for Fmoc deprotection is to stir the Fmoc-peptide in a solution of dimethylformamide (DMF) or dichloromethane (DCM) with excess piperidine, deprotecting the Fmoc group and forming NFMP in the process. The '891 patent teaches removal of this impurity by deprotecting with 4-aminomethylpiperidine (4AMP) instead of piperidine. This forms NFMP-CH₂NH₂ instead of NFMP, which due to the presence of the extra amino group, can be extracted into water. The 4AMP additionally scavenges the active amino acid ester and enables the removal of the 4AMP-amino-acid product from the reaction mixture via washes. A significant problem with this method lies in the high cost of using 4AMP, rendering the method cost prohibitive and preventing it from being widely accepted by the industry.

Another example of Fmoc-based SolPPS can be seen in published patent application WO2017112809A1. This publication teaches the use of a C-terminus GAP protecting group, benzyl diphenylphosphine oxide (HOBnDpp), to control the solubility of the target peptide to allow for selective precipitation after each successive coupling reaction. The solubility is controlled such that the growing peptide remains in an organic solvent, such as ethyl acetate or DCM, and aqueous washes are performed to remove impurities; a subsequent concentration of the organic solvent followed by mixture in an alkane solvent selectively precipitates the peptide product. While this technology adapted Fmoc/tBu chemistry to solution-phase in a much more economically feasible manner than the '891 patent, there are potential limitations inherent in the method, such as challenges in removing the GAP protecting group from the peptide; maintaining solubility control over longer sequences; accidental cleavage of the GAP protecting group during the coupling/deprotection reactions; inability to easily perform C-terminal modifications; and racemization, such as at the point of coupling between the GAP protecting group and the peptide.

### SUMMARY OF THE INVENTION

The present disclosure offers several advantages in the art. In one embodiment, a novel method of solution-phase peptide synthesis is presented that utilizes a modular approach to anchor synthesis that can increase solubility control over a given peptide sequence. In another embodiment, the present disclosure includes using a peptide as an anchor to synthesize a target peptide. Preferably, in one embodiment, the peptide can include functionalized protecting groups that can assist in solubility.

In one embodiment, the present disclosure can include a chemical composition comprising a GAP constituent, a stopper constituent, and a linker constituent. In another embodiment, the present disclosure can include a method of solution-phase peptide synthesis, and the method can include the steps of attaching a first peptide to a first amino acid; coupling one or more additional amino acids to the first amino acid to form a second peptide; and removing the first peptide from the second peptide.

In another embodiment, the present disclosure can include a method of forming an anchor peptide for solution-phase peptide synthesis, and the method can include the steps of coupling a first protected amino acid to a second protected amino acid; and attaching a linker to either the first or the second protected amino acid.

In another embodiment, the present disclosure can include a chemical composition, which can be selected from the group consisting of:

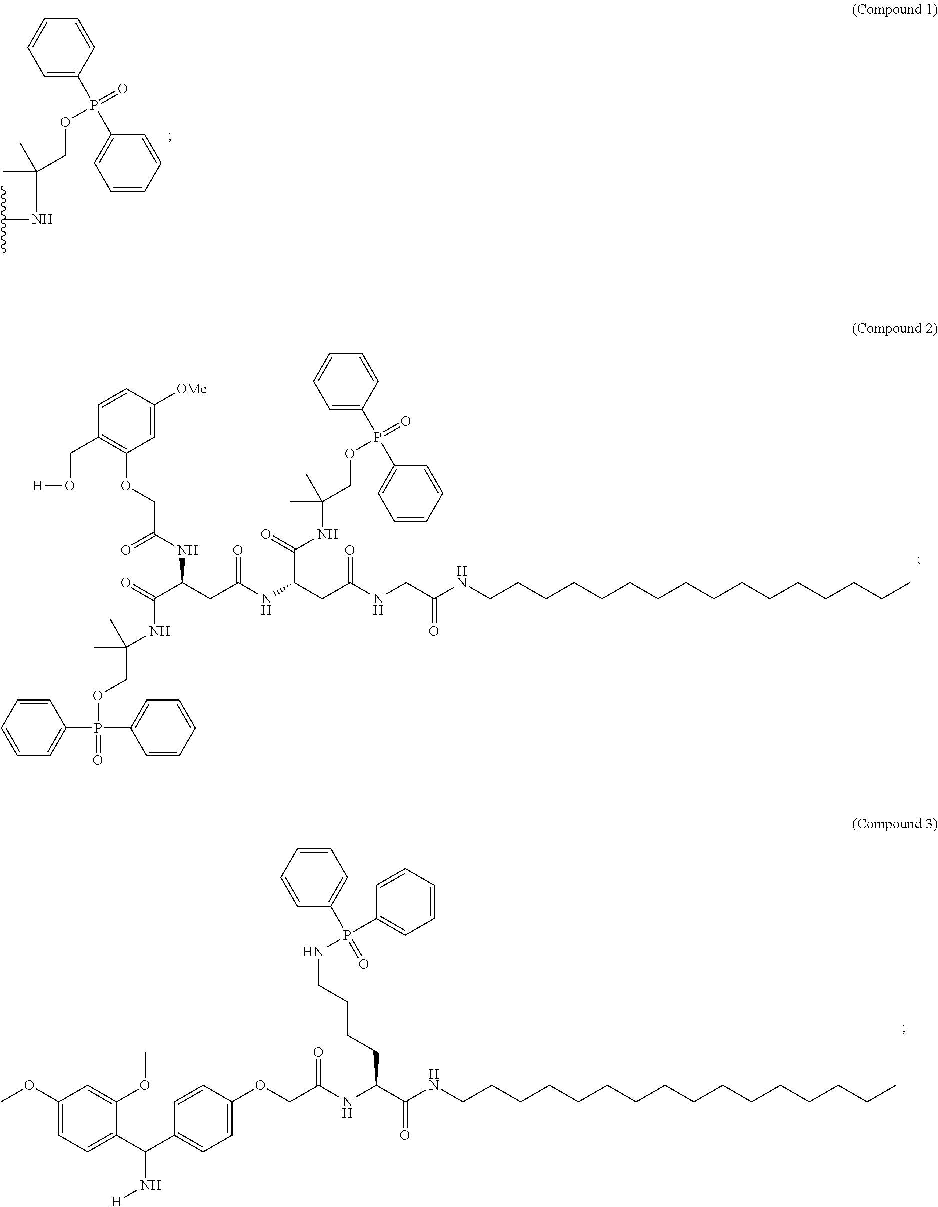

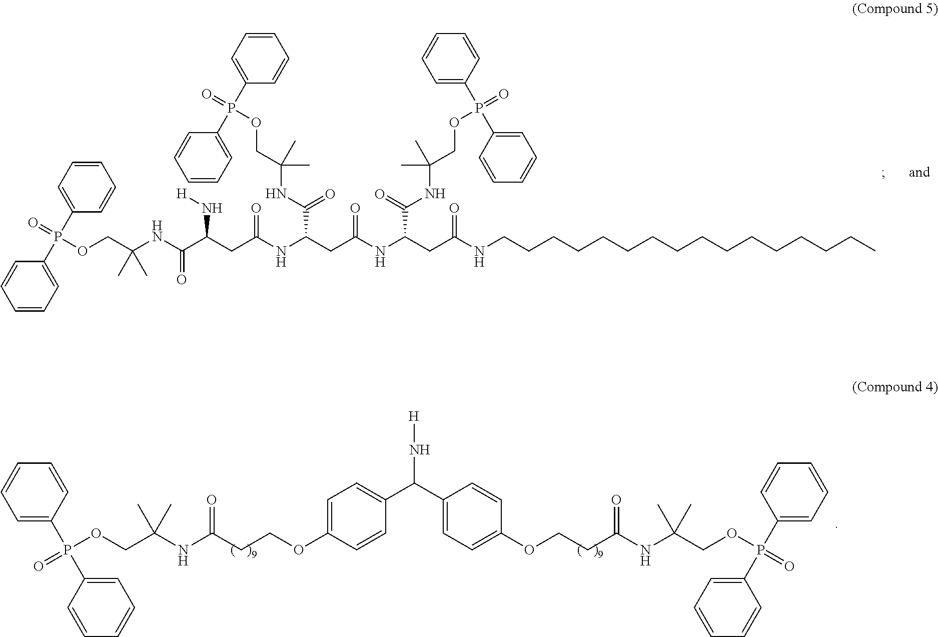

In another embodiment, the present disclosure can include a method of peptide synthesis, and the method can include attaching a first protecting group to a first side chain of a first amino acid; coupling a second protecting group to a second side chain of a second amino acid; coupling the first amino acid with the second amino acid; attaching a linker to a terminus of the first or second amino acid; attaching the linker to a third amino acid; and coupling a fourth amino acid with the third amino acid.

In another embodiment, the present disclosure can include a chemical composition comprising a GAP constituent selected from the group consisting of:

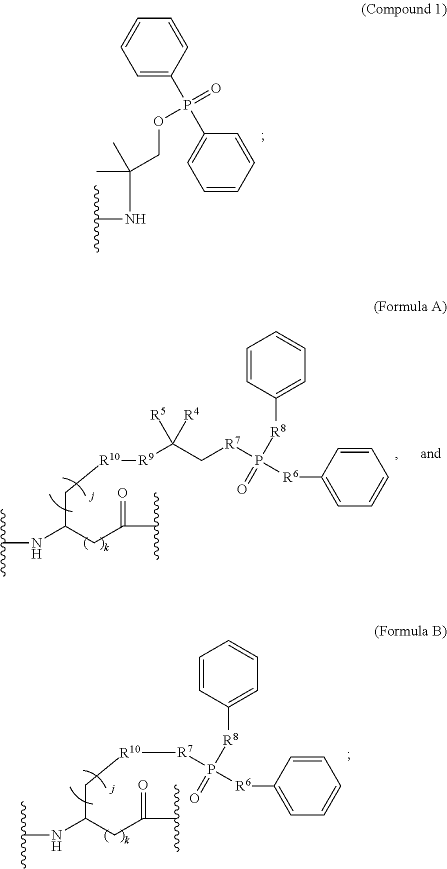

wherein: R⁴ is selected from the group consisting of: H, methyl, ethyl, propyl, isopropyl, benzyl, isobutyl, sec-butyl, and tert-butyl; R⁵ is selected from the group consisting of: H, methyl, ethyl, propyl, isopropyl, benzyl, isobutyl, sec-butyl, and tert-butyl; R⁶ is selected from the group consisting of: —C(O)—, S, O, NH, NMe, Net, NBn, NPh, and —(CH₂)ₚ—, wherein “p1” is an integer selected from the range of 0 up to and including 30; R⁷ is selected from the group consisting of: —C(O)—, S, O, NH, NMe, Net, NBn, NPh, and —(CH₂)ₚ₂—, wherein “p2” is an integer selected from the range of 0 up to and including 30; R⁸ is selected from the group consisting of: —C(O)—, S, O, NH, NMe, Net, NBn, NPh, and —(CH₂)ₚ₃—, wherein “p3” is an integer selected from the range of 0 up to and including 30; R⁹ is selected from the group consisting of: —C(O)—, S, O, NH, NMe, Net, NBn, NPh, and —(CH₂)ₚ₄—, wherein “p4” is an integer selected from the range of 0 up to and including 30; R¹⁰ is selected from the group consisting of: —C(O)—, —(CH₂)ₘ—, and NH, wherein “m” is an integer selected from the range of 0 up to and including 30; “j” is an integer selected from the range of 0 up to and including 30; and “k” is an integer selected from the range of 0 up to and including 30. Further comprising a linker constituent, wherein the linker constituent is selected from the group consisting of:

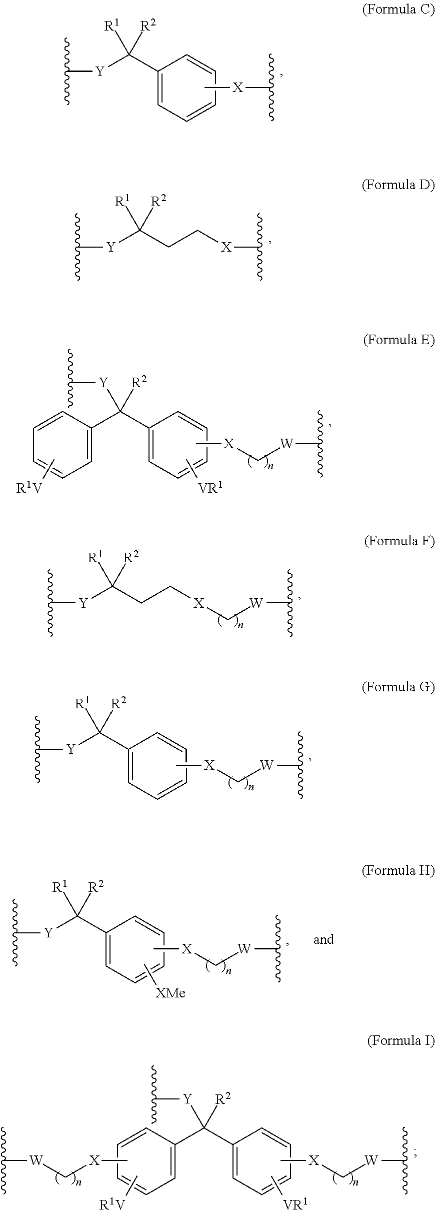

wherein Y is selected from the group consisting of: S, NH, NMe, NEt, NBn, NPh, and O; Vis selected from the group consisting of: S, NH, NMe, NEt, NBn, NPh, O, —C(O)—, —(CH₂)ₐ₁—, wherein “a1” is an integer selected from the range of 0 up to and including 30; W is selected from the group consisting of: S, NH, NMe, NEt, NBn, NPh, O, —C(O)—, —(CH₂)ₐ₂—, wherein “a2” is an integer selected from the range of 0 up to and including 30; X is selected from the group consisting of: S, NH, NMe, NEt, NBn, NPh, O, —C(O)—, —(CH₂)ₐ₃—, wherein “a3” is an integer selected from the range of 0 up to and including 30; R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30; R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and “n” is an integer selected from the range of 0 up to and including 30. Further comprising a stopper constituent, wherein the stopper constituent is selected from the group consisting of:

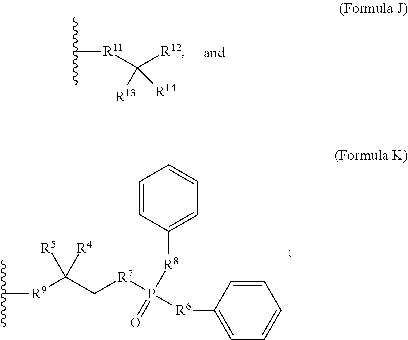

wherein R⁴ is selected from the group consisting of: H, methyl, ethyl, propyl, isopropyl, benzyl, isobutyl, sec-butyl, and tert-butyl; R⁵ is selected from the group consisting of: H, methyl, ethyl, propyl, isopropyl, benzyl, isobutyl, sec-butyl, and tert-butyl; R⁶ is selected from the group consisting of: —C(O)—, S, O, NH, NMe, Net, NBn, NPh, and —(CH₂)_{q1}—, wherein “q1” is an integer selected from the range of 0 up to and including 30; R⁷ is selected from the group consisting of: —C(O)—, S, O, NH, NMe, Net, NBn, NPh, and —(CH₂)_{q2}—, wherein “q2” is an integer selected from the range of 0 up to and including 30; R⁸ is selected from the group consisting of: —C(O)—, S, O, NH, NMe, Net, NBn, NPh, and —(CH2)_{q3}—, wherein “q3” is an integer selected from the range of 0 up to and including 30; R⁹ is selected from the group consisting of: —C(O)—, S, O, NH, NMe, Net, NBn, NPh, and —(CH2)_{q4}—, wherein “q4” is an integer selected from the range of 0 up to and including 30; R¹¹ is selected from the group consisting of: S, O, NH, NMe, Net, NBn, and NPh; R¹² is selected from the group consisting of: H, methyl, ethyl, propyl, isopropyl, benzyl, isobutyl, sec-butyl, tert-butyl, and —(CH₂)_{q5}—H, wherein “q5” is an integer selected from the range of 0 up to and including 30; R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30. Further comprising a linker constituent, a stopper constituent, and a first amino acid constituent, wherein the GAP constituent is

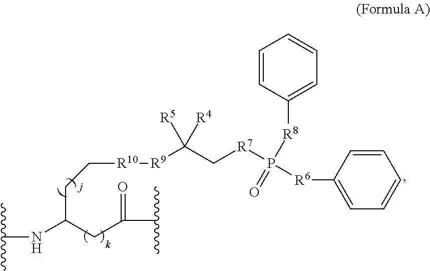

wherein R⁴ is selected from the group consisting of: H, methyl, ethyl, propyl, isopropyl, benzyl, isobutyl, sec-butyl, and tert-butyl; R⁵ is selected from the group consisting of: H, methyl, ethyl, propyl, isopropyl, benzyl, isobutyl, sec-butyl, and tert-butyl; R⁶ is selected from the group consisting of: —C(O)—, S, O, NH, NMe, Net, NBn, NPh, and —(CH₂)ₚ₁—, wherein “p1” is an integer selected from the range of 0 up to and including 30; R⁷ is selected from the group consisting of: —C(O)—, S, O, NH, NMe, Net, NBn, NPh, and —(CH₂)ₚ₂—, wherein “p2” is an integer selected from the range of 0 up to and including 30; R⁸ is selected from the group consisting of: —C(O)—, S, O, NH, NMe, Net, NBn, NPh, and —(CH₂)ₚ₃—, wherein “p3” is an integer selected from the range of 0 up to and including 30; R⁹ is selected from the group consisting of: —C(O)—, S, O, NH, NMe, Net, NBn, NPh, and —(CH₂)ₚ₄—, wherein “p4” is an integer selected from the range of 0 up to and including 30; R¹⁰ is selected from the group consisting of: —C(O)—, —(CH₂)ₘ—, and NH, wherein “m” is an integer selected from the range of 0 up to and including 30; “j” is an integer selected from the range of 0 up to and including 30; and “k” is an integer selected from the range of 0 up to and including 30; the linker constituent is

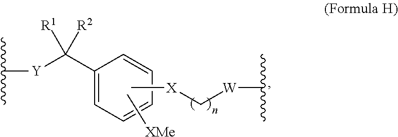

wherein: Y is selected from the group consisting of: S, NH, NMe, NEt, NBn, NPh, and O; W is —C(O)—; X is selected from the group consisting of: S, NH, NMe, NEt, NBn, NPh, O, —C(O)—, —(CH₂)ₐ₃—, wherein “a3” is an integer selected from the range of 0 up to and including 30; R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30; R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and “n” is an integer selected from the range of 0 up to and including 30; the first amino acid constituent is

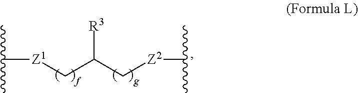

wherein Z¹ is selected from the group consisting of: S, O, NH, NMe, Net, NBn, NPh, —C(O)—, and —(CH₂)_{d}—, wherein “d” is an integer selected from the range of 0 up to and including 30; Z² is selected from the group consisting of: S, O NH, NMe, Net, NBn, NPh, —C(O)—, and —(CH₂)ₑ—, wherein “e” is an integer selected from the range of 0 up to and including 30; “f” is an integer selected from the range of 0 up to and including 30; “g” is an integer selected from the range of 0 up to and including 30; R³ is selected from the group consisting of: H, methyl, ethyl, propyl, isopropyl, benzyl, isobutyl, sec-butyl, tert-butyl, 2-(methylmercapto)ethyl,

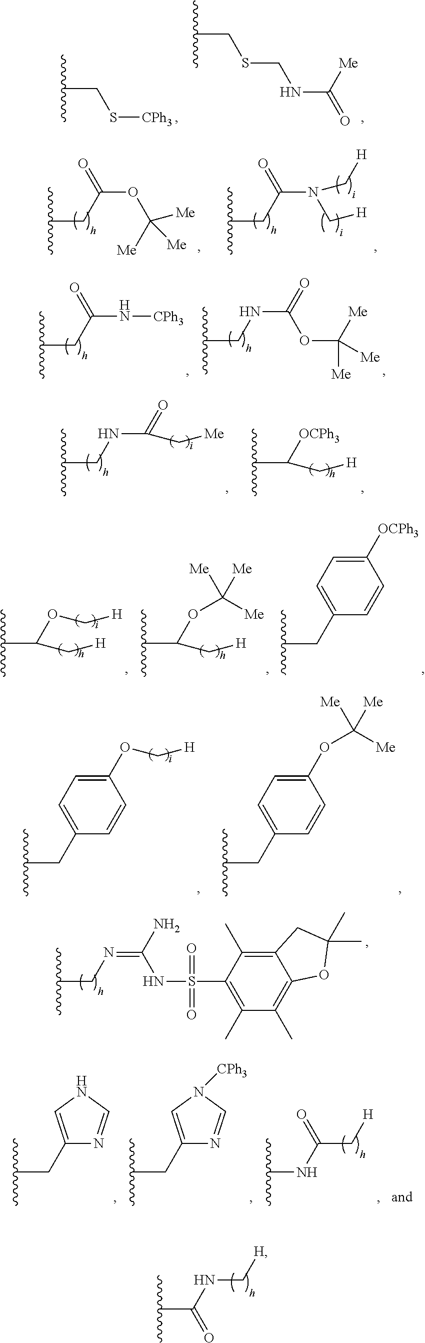

wherein “h” is an integer selected from the range of 0 up to and including 30; and the stopper constituent is

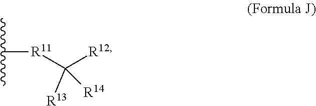

wherein R¹¹ is selected from the group consisting of: S, O, NH, NMe, Net, NBn, and NPh; R¹² is selected from the group consisting of: H, methyl, ethyl, propyl, isopropyl, benzyl, isobutyl, sec-butyl, tert-butyl, and —(CH₂)_{q5}—H, wherein “q5” is an integer selected from the range of 0 up to and including 30; R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30. Comprising:

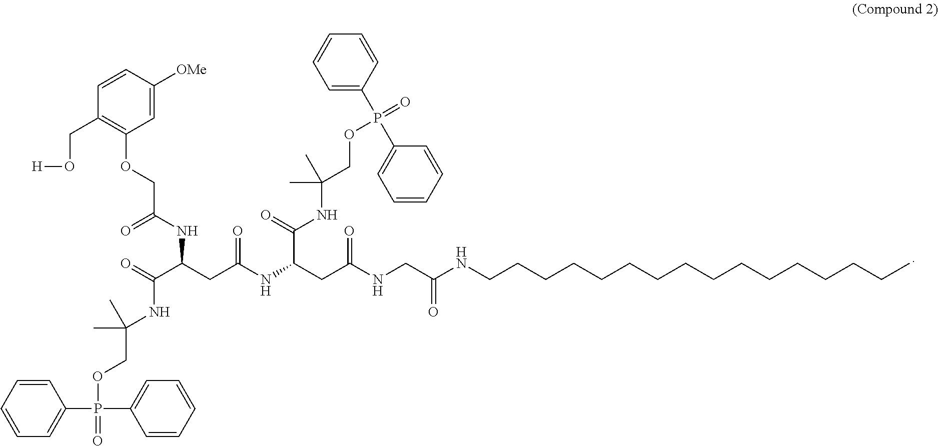 [SMILES: CCCCCCCCCCCCCCCCNC(=O)CNC(=O)C[C@H](NC(=O)C[C@H](NC(=O)COc1cc(OC)ccc1CO)C(=O)NC(C)(C)COP(=O)(c1ccccc1)c1ccccc1)C(=O)NC(C)(C)COP(=O)(c1ccccc1)c1ccccc1]

In another embodiment, the present disclosure can include a chemical composition comprising a GAP constituent, a stopper constituent, and a linker constituent, wherein the GAP constituent is

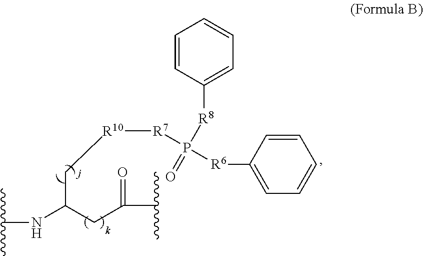

wherein: R⁶ is selected from the group consisting of: —C(O)—, S, O, NH, NMe, Net, NBn, NPh, and —(CH₂)ₚ₁—, wherein “p1” is an integer selected from the range of 0 up to and including 30; R⁷ is selected from the group consisting of: —C(O)—, S, O, NH, NMe, Net, NBn, NPh, and —(CH₂)ₚ₂—, wherein “p2” is an integer selected from the range of 0 up to and including 30; R⁸ is selected from the group consisting of: —C(O)—, S, O, NH, NMe, Net, NBn, NPh, and —(CH₂)ₚ₃—, wherein “p3” is an integer selected from the range of 0 up to and including 30; R¹⁰ is selected from the group consisting of: —C(O)—, —(CH₂)ₘ—, and NH, wherein “m” is an integer selected from the range of 0 up to and including 30; “j” is an integer selected from the range of 0 up to and including 30; and “k” is an integer selected from the range of 0 up to and including 30; the linker constituent is

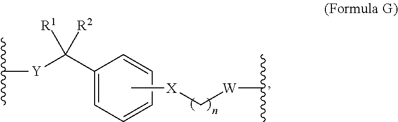

wherein Y is selected from the group consisting of: S, NH, NMe, NEt, NBn, NPh, and O; W is —C(O)—; X is selected from the group consisting of: S, NH, NMe, NEt, NBn, NPh, O, —C(O)—, —(CH₂)ₐ₃—, wherein “a3” is an integer selected from the range of 0 up to and including 30; R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30; R² is selected from the group consisting of —(CH₂)_{c}—H, —CCl₃, —CF3, phenyl, isopropyl, tert-butyl, chlorophenyl, dichlorophenyl, methoxyphenyl, and dimethoxyphenyl, wherein “c” is an integer selected from the range of 0 up to and including 30; and “n” is an integer selected from the range of 0 up to and including 30; and the stopper constituent is

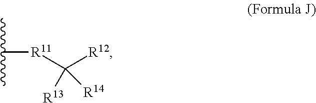

wherein R¹¹ is selected from the group consisting of: S, O, NH, NMe, Net, NBn, and NPh; ¹² is selected from the group consisting of: H, methyl, ethyl, propyl, isopropyl, benzyl, isobutyl, sec-butyl, tert-butyl, and —CH₂)_{q5}—H, wherein “q5” is an integer selected from the range of 0 up to and including 30; R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30. Comprising:

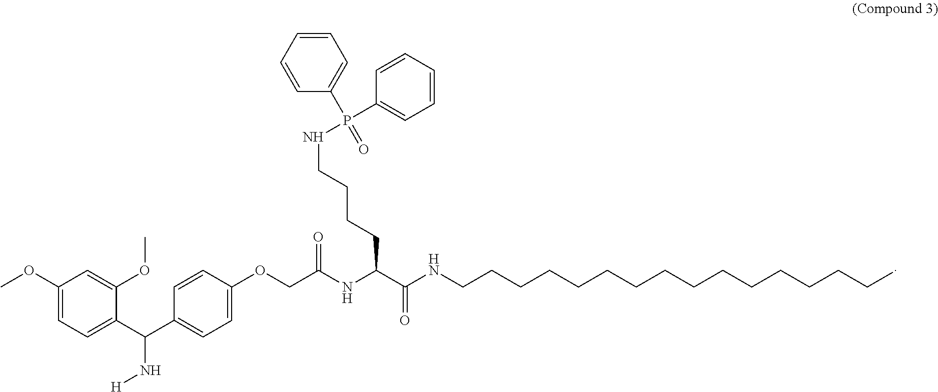

In another embodiment, the present disclosure can include a chemical composition comprising a GAP constituent, a stopper constituent, and a linker constituent, wherein the GAP constituent is

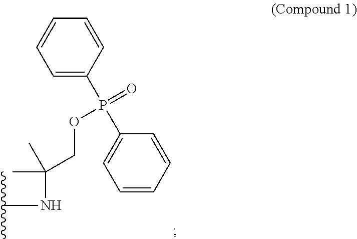 [SMILES: *NC(C)(C)COP(=O)(c1ccccc1)c1ccccc1]

linker constituent is

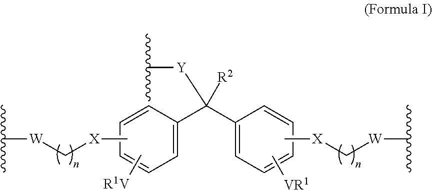

wherein Y is selected from the group consisting of: S, NH, NMe, NEt, NBn, NPh, and O; V is selected from the group consisting of: S, NH, NMe, NEt, NBn, NPh, O, —C(O)—, —(CH₂)ₐ₁—, wherein “a1” is an integer selected from the range of 0 up to and including 30; W is selected from the group consisting of: S, NH, NMe, NEt, NBn, NPh, O, —C(O)—, —(CH₂)ₐ₂—, wherein “a2” is an integer selected from the range of 0 up to and including 30; X is selected from the group consisting of: S, NH, NMe, NEt, NBn, NPh, O, —C(O)—, —(CH₂)ₐ₃—, wherein “a3” is an integer selected from the range of 0 up to and including 30; R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30; R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and “n” is an integer selected from the range of 0 up to and including 30; and the stopper constituent is

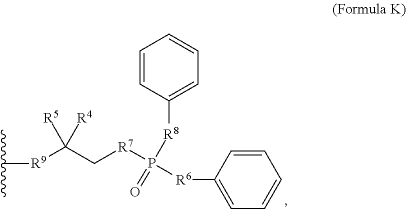

wherein R⁴ is selected from the group consisting of: H, methyl, ethyl, propyl, isopropyl, benzyl, isobutyl, sec-butyl, and tert-butyl; R⁵ is selected from the group consisting of: H, methyl, ethyl, propyl, isopropyl, benzyl, isobutyl, sec-butyl, and tert-butyl; R⁶ is selected from the group consisting of: —C(O)—, S, O, NH, NMe, Net, NBn, NPh, and —(CH2)_{q1}-, wherein “q1” is an integer selected from the range of 0 up to and including 30; R⁷ is selected from the group consisting of: —C(O)—, S, O, NH, NMe, Net, NBn, NPh, and —(CH2)_{q2}-, wherein “q2” is an integer selected from the range of 0 up to and including 30; R⁸ is selected from the group consisting of: —C(O)—, S, O, NH, NMe, Net, NBn, NPh, and —(CH2)_{q3}-, wherein “q3” is an integer selected from the range of 0 up to and including 30; and R⁹ is selected from the group consisting of: —C(O)—, S, O, NH, NMe, Net, NBn, NPh, and —(CH2)_{q4}-, wherein “q4” is an integer selected from the range of 0 up to and including 30. Comprising

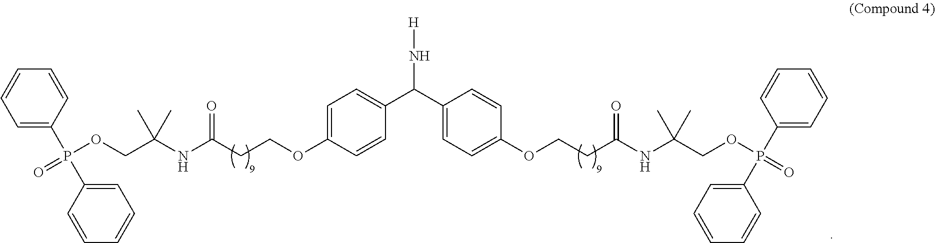

In another embodiment, the present disclosure can include a method of solution-phase peptide synthesis, the method comprising the steps of: attaching a first peptide to a first amino acid, wherein the first peptide is an anchor peptide; coupling one or more additional amino acids to the first amino acid to form a second peptide; and removing the first peptide from the second peptide. Wherein the first peptide includes a GAP constituent. Wherein the first peptide is formed by coupling a first protected amino acid to a second protected amino acid and attaching a linker to either the first or the second protected amino acid, wherein the first protected amino acid is formed by attaching a first protecting group to a first side chain of a first amino acid constituent, and the second protected amino acid is formed by coupling a second protecting group to a second side chain of a second amino acid constituent. Further comprising the steps of: coupling the first amino acid constituent with the second amino acid constituent; attaching the linker to a terminus of the first or second amino acid constituent; attaching the linker to a third amino acid; and coupling a fourth amino acid with the third amino acid.

In another embodiment, the present disclosure can include a method of peptide synthesis, comprising the steps of coupling an anchor to a first amino acid, and coupling a second amino acid with the first amino acid, wherein the anchor includes a compound selected from the group consisting of:

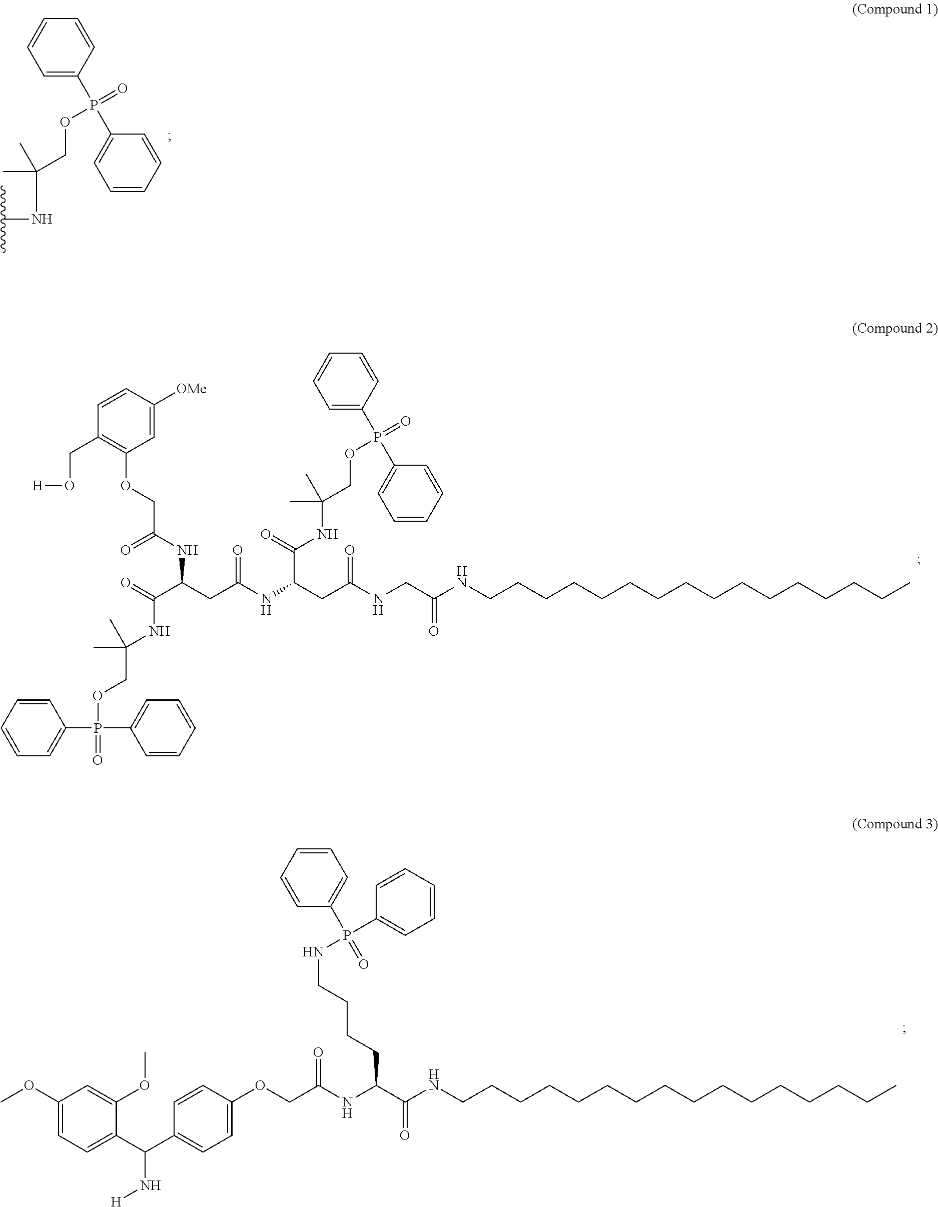

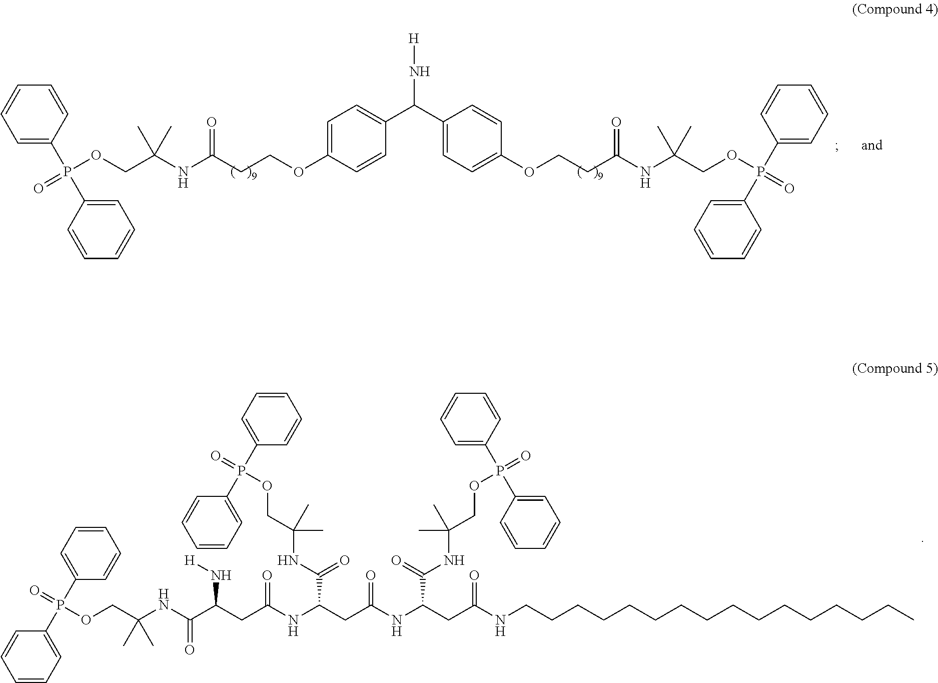

Wherein the anchor is coupled to a C-terminus of the first amino acid. Wherein the anchor is coupled to a side chain of the first amino acid. wherein the compound is

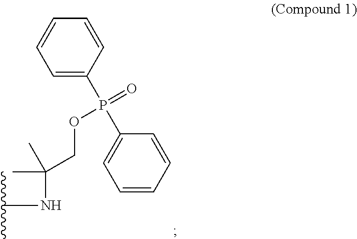 [SMILES: *NC(C)(C)COP(=O)(c1ccccc1)c1ccccc1]

and wherein the anchor is coupled to the C-terminus of the first amino acid via a linker constituent. Wherein the anchor further includes a stopper constituent. Further comprising the step of removing the anchor from the first amino acid. Wherein the coupling of the second amino acid with the first amino acid occurs in 2-methyltetrahydrofuran.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure. The figures are used as non-limiting examples, only intended to portray preferred embodiments without limiting the scope of this disclosure:

FIG. 1A illustrates an exemplary GAP constituent in accordance with the principles of the present disclosure;

FIG. 1B illustrates exemplary AA(Gap) constituents in accordance with the principles of the present disclosure;

FIG. 2 illustrates exemplary amino acid constituents in accordance with the principles of the present disclosure;

FIG. 3 illustrates exemplary linker constituents in accordance with the principles of the present disclosure;

FIG. 4 illustrates exemplary stopper constituents in accordance with the principles of the present disclosure;

FIG. 5 illustrates an exemplary anchor molecule and/or anchor peptide with an exemplary underlying structural schematic in accordance with the principles of the present disclosure;

FIG. 6 illustrates an exemplary anchor molecule with an exemplary underlying structural schematic in accordance with the principles of the present disclosure;

FIG. 7 illustrates an exemplary anchor molecule and/or anchor peptide with an exemplary underlying structural schematic in accordance with the principles of the present disclosure; and

FIG. 8 illustrates an exemplary modular anchor synthesis method in accordance with the principles of the present disclosure.

### DETAILED DESCRIPTION OF THE DISCLOSURE

In the Summary of the Disclosure above and in the Detailed Description of the Disclosure, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference if made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(an initial number) to (a subsequent number)” or “(an initial number)-(a subsequent number),” this means a range whose lower limit is the initial number and whose upper limit is the subsequent number. For example, 25 to 100 mm means a range whose lower limit is 25 mm, and whose upper limit is 100 mm.

“Amino acid” is a term commonly understood in the art. An amino acid, in the broadest sense, can be any organic molecule containing both an amino group and a carboxylic acid group. For practical application, these molecules can generally be less than 1,000 Daltons in size, with the amino group (N-terminus) separated from the carboxylic acid group (C-terminus) by only one or two methylene units. An additional feature of an amino acid can be the presence of a side chain, as a branch from the methylene unit separating the N and C termini. The side chain can include practically any structural feature, and the variation in the side chain accounts for the variation in amino acids. This variation includes, but is not limited to, the standard twenty proteinogenic amino acids, along with others such as ornithine or selenocysteine. Many non-proteinogenic structures can also be considered amino acids, such as phenylglycine, and so the term is not limited to a subset of twenty. “Protected” amino acids can be considered a sub-category of amino acids where certain functional groups, such as the N-terminus and/or portions of the side chain as non-limiting examples, are attached to protecting groups to temporarily inhibit the reactivity of the protected functionality. In the context of this disclosure, “GAP amino acids” can be amino acids where the side chain and/or C-terminus and/or N-terminus contains and/or is attached to one or more GAP groups. “GAP groups” as used herein can include GAP constituents (GAP anchors) (GAP protecting groups) as further discussed below. GAP groups can include, but are not limited to, a phosphine oxide moiety.

As mentioned above, the amino acid “side chain” can be a branch from the methylene unit separating the N and C termini, and can also sometimes be referred to generally as an “R” group. A side chain can include practically any structural feature, and the variation in the side chain accounts for the variation in amino acids.

“Peptide” is a term commonly understood in the art as a biopolymer made from amino acid monomers. There must be at least two amino acids bonded together to constitute a peptide. An “anchor peptide” as used herein can include a peptide that also functions as an anchor molecule—for anchor peptides, a peptide is made, which then can function as an anchor to synthesize the second, target peptide. For the purposes of this disclosure, an anchor peptide can include a peptide having 20 or fewer amino acids. An “anchor” as used herein can be a molecular group that is attached or designed for attachment to a target molecule (such as a peptide) for the purpose of controlling the solubility properties of that peptide during its synthesis, in order to facilitate the synthesis of the peptide in a practical fashion. Anchors can generally be removed at the end of the peptide synthesis and may not be a component of the final target molecule. However, upon removal of an anchor, in some embodiments, a molecular component of the anchor may remain, such as to accomplish a, e.g., C-terminal modification (such as may be accomplished via the know Rink Amide linker). A “GAP anchor” as used herein can include an anchor molecule that includes a GAP group. An anchor can include a GAP constituent (discussed below). In one embodiment, a GAP constituent can itself serve as an anchor.

In one embodiment, the present disclosure can include structures of anchor molecule for Group-Assisted Purification Peptide Synthesis (GAP-PS). In one embodiment, an anchor molecule can be coupled to a first amino acid in a peptide sequence without causing significant racemization. In another embodiment, an anchor molecule can be stable to the conditions of amino acid coupling and Fmoc deprotection steps. In another embodiment, an anchor molecule can be cleaved from the peptide without causing racemization, either concomitantly with the side-chain protecting groups, or via an orthogonal method. In another embodiment, the anchor can provide substantial control over solubility. For example, in one embodiment, an anchor molecule can completely exclude a protected peptide from dissolving in water or water/organic solvent mixtures where the water content is >50%. In one embodiment, failure of an anchor molecule to do this could result in low yields due to repetitive loss of product during aqueous workup. In another embodiment, an anchor can also promote solubility of a protected peptide in the chosen organic solvent, such that the peptide can completely dissolve to form a homogenous solution at concentrations suitable for large-scale manufacture, typically on the order of 10-20 mL of solvent per gram of solute. In another embodiment, can anchor can be a protection group capable for “anchoring” a target molecule in a particular solvent.

In another embodiment, the present disclosure can include anchors that provide significant improvement to these above-discussed properties, as well as providing additional benefits. For example, the anchors considered herein can include a GAP-protected amino acid, and in another example, the utilization of such GAP-protected amino acid to build, in a modular fashion, an anchor that can be custom-tailored to a peptide being synthesized. In another embodiment, the present disclosure can include a system that can use fast and reliable chemistry of peptide coupling and deprotection reactions to build an anchor (GAP anchor). In some embodiments, a GAP anchor can include an anchor peptide (GAP anchor peptide). In one embodiment, an anchor peptide can be an anchor having a peptide bond; in another embodiment, an anchor peptide can be any peptide; in another embodiment, an anchor peptide can be any molecule having a peptide bond that is soluble in polar organic solvents and insoluble in water. In another embodiment, an anchor peptide can be any molecule having a peptide bond that can be removably attached to a target molecule to facilitate solubility control, and subsequently removed from the target molecule. In another embodiment, anchors can be quickly synthesized that incorporate one, two, three, or more GAP amino acids for varying degrees of control over solubility that, for example, can be balanced with cost. For example, synthesis of a 30-mer peptide can be accomplished via the use of three GAP amino acids in the anchor to adequately control the solubility of a peptide this size; however, in another example, a 5-mer peptide can be achieved using an anchor with one GAP amino acid. In another example, the same GAP amino acid can be synthesized as a common feedstock for both of these anchors, and can be quickly assembled as an anchor in a manner consistent with the needs of the target molecule, and in a way which balances the control required with the cost of anchor synthesis and atom economy.

In one embodiment, and as a result of increased stability, additional process advantages can be achieved. In another embodiment, the increased stability of the GAP amino acid anchors can allow both the coupling and deprotection reactions to be run in wet organic solvent (water content <10%). This can, for example, eliminate the need for time-consuming, and therefore costly, drying steps which can include filtration through a solid drying agent and/or azeotropic distillation. In another embodiment, the present disclosure offers significant advantages by eliminating the need for these processing steps, the entire synthesis can be significantly faster, meaning more coupling cycles can be achieved in a shorter period. Such advantages can have a significant, positive effect on the overhead allocation of the peptide synthesis cost, especially for longer peptides where more coupling cycles are required.

In one embodiment, the present disclosure can include the utilization of amino acids and/or peptides in combination with protecting groups to form anchor molecules for solution-phase peptide synthesis. For example, amino acids can be protected at their side chains and/or termini with protecting groups (e.g. GAP protecting groups), and further coupled to one another (and/or to other molecules) to form anchor molecules.

In one embodiment, the present disclosure can include a chemical composition that can include one or more group-assisted purification (GAP) constituents (in one embodiment, each GAP constituent can preferably be less than 300 Daltons); one or more amino acid (AA) constituents; one or more linker constituents; and/or one or more stopper (cap) constituents. In another embodiment, a GAP constituent can be an amino-acid-GAP constituent (AA(Gap) constituent). In one embodiment, a GAP constituent and linker constituent can be GAP constituents and linker constituents (respectively) as described in U.S. application Ser. No. 17/104,166. In another embodiment, a stopper constituent can be similar to or the same as the spacer constituents discussed in U.S. application Ser. No. 17/104,166. In one embodiment, the present disclosure can include structures of the type H-AA(Gap)-OH, H-[AA¹]_{α}-[AA(Gap)]_{β}-[AA²]_{γ}-Stopper, H-Linker-[AA¹]_{α}-[AA(Gap)]_{β}-[AA²]_{γ}-Stopper, and/or H-Linker-{[AA1]_{α}-[AA(Gap)]_{β}-[AA2]_{γ}-Stopper}_{δ}, which can collectively be referred to as H-Anchor (anchor) (GAP anchor) molecules, where H can refer to the element hydrogen; O can refer to the element oxygen; α, β, and γ can be any integer from 0 up to and including 10; δ can be any integer from 0 up to and including 2; AA(Gap) can be an amino-acid-GAP constituent (e.g., an amino acid attached to a GAP constituent); Linker can be a linker constituent like those discussed herein and known in the art; AA¹ and/or AA² can be any type of amino acid or amino acid moiety; and Stopper can be, e.g., any chemical moiety which is largely chemically inert, has very low solubility in water, does not hinder the solubility of the anchor molecule in organic solvent, and contains a nucleophilic portion at one end such that the other chemical moieties and/or constituents of the anchor molecule can be attached using standard peptide coupling reagents.

Any variables used herein that can have similar values are not required to be the same value. For example, and in one embodiment, R⁴ and R⁵ can both be any of H, methyl, ethyl, propyl, isopropyl, benzyl, isobutyl, sec-butyl, or tert-butyl—they can be the same or different. Further,

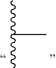

can depict an attachment point of a given molecule to any other molecule.

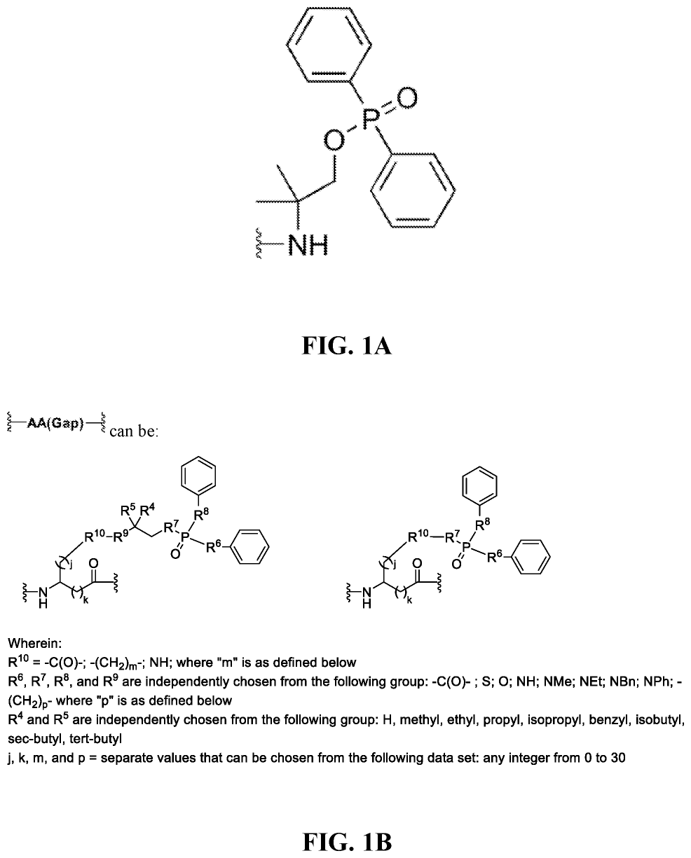

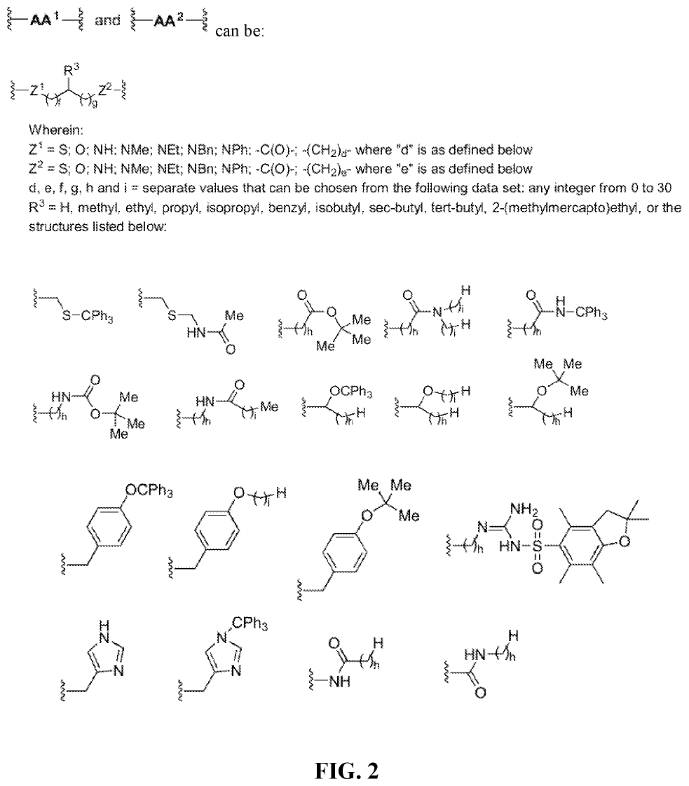

For example, and in another embodiment exemplified in FIG. 1A, a GAP constituent can include:

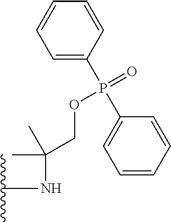 [SMILES: *NC(C)(C)COP(=O)(c1ccccc1)c1ccccc1]

(HNtBuODpp) wherein

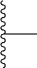

can depict attachment of this GAP constituent to another molecule or atom, such as to an amino acid (e.g. with respect to an AA(Gap) constituent). For example, and in one embodiment, HNtBuODpp (or another GAP constituent) can be coupled to an amino acid side-chain or C-terminus to form an AA(Gap) constituent. In one embodiment, attaching a GAP constituent to an amino acid side chain or C-terminus can leave the C-terminus or side chain (respectively) of the amino acid free to participate in coupling reactions, such as with additional GAP-protected amino acids. Preferably, one or more AA(Gap) constituents can be included in an anchor molecule, or be an anchor molecule. In another embodiment, two or more AA(Gap) constituents can be coupled together, such as to form an anchor molecule or part of an anchor molecule. In one example, by utilizing two or more AA(Gap) constituents, a peptide-based anchor molecule can be formed to enable solution-phase peptide synthesis.

In another embodiment, a GAP constituent can be an AA(Gap) constituent and can preferably include an amino acid coupled to a group-assisted purification molecule. For example, an AA(Gap) constituent can include a GAP constituent and an amino acid constituent. In another embodiment, an AA(Gap) constituent

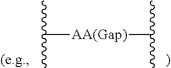

can include the following structures as exemplified in FIG. 1B (such as with respect to anchors including H-AA(Gap)-OH, H-[AA¹]_{α}-[AA(Gap)]_{β}-[AA²]_{γ}-Stopper, H-Linker-[AA¹]_{α}-[AA(Gap)]_{β}-[AA²]_{γ}-Stopper), and/or H-Linker-{[AA1]_{α}-[AA(Gap)]_{β}-[AA2]_{γ}-Stopper}_{δ}:

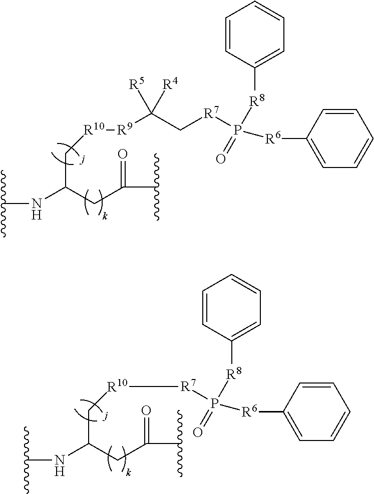

wherein R¹⁰ can be —C(O)—, —(CH₂)ₘ—, or NH; R⁶, R⁷, R⁸, and R⁹ can be any of —C(O)—, S, O, NH, NMe, Net, NBn, NPh, or —(CH₂)ₚ—; R⁴ and R⁵ can be H, methyl, ethyl, propyl, isopropyl, benzyl, isobutyl, sec-butyl, or tert-butyl; and j, k, m, and p can each be separate a value that can be any integer from 0 up to and including 30. R⁶, R⁷, R⁸, and R⁹ can all be different, and “p” can be a different value for each of R⁶, R⁷, R⁸, and R⁹.

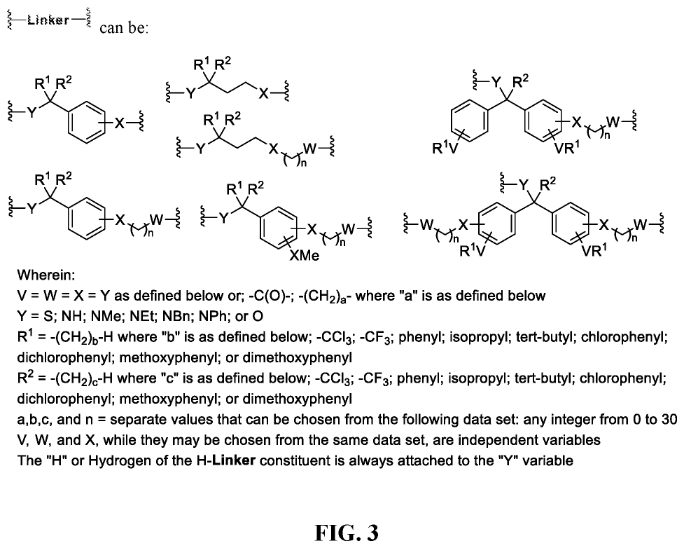

In another embodiment, an anchor can include an amino acid constituent (AA constituent), such as in addition to a GAP constituent or AA(Gap) constituent, such as with respect to anchors including H-AA(Gap)-OH, H-[AA¹]_{α}-[AA(Gap)]_{β}-[AA²]_{γ}-Stopper, and/or H-Linker-[AA¹]_{α}-[AA(Gap)]_{β}-[AA²]_{γ}-Stopper. In one embodiment, an amino acid constituent can be any molecule having a primary or secondary amine, and a carboxylic acid. In another embodiment, and as illustrated in FIG. 2, an amino acid constituent

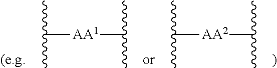

can include:

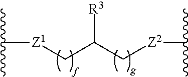

wherein Z¹ can be S, O, NH, NMe, Net, NBn, NPh, —C(O)—, or —(CH₂)_{d}—; Z² can be S, O NH, NMe, Net, NBn, NPh, —C(O)—, or —(CH₂)ₑ—; d, e, f, g, h, can each be a value that can be any integer from 0 up to and including 30; and R³ can be H, methyl, ethyl, propyl, isopropyl, benzyl, isobutyl, sec-butyl, tert-butyl, 2-(methylmercapto)ethyl, or:

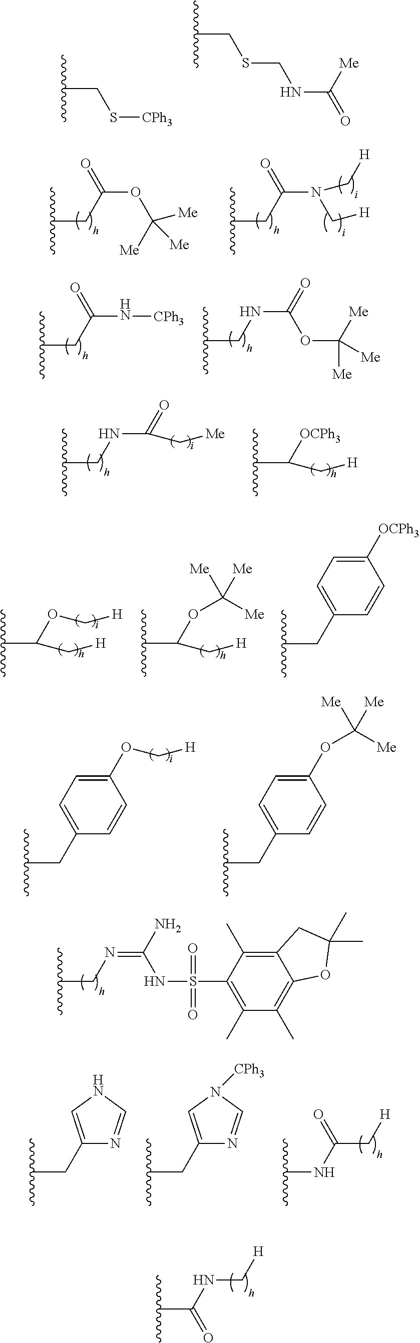

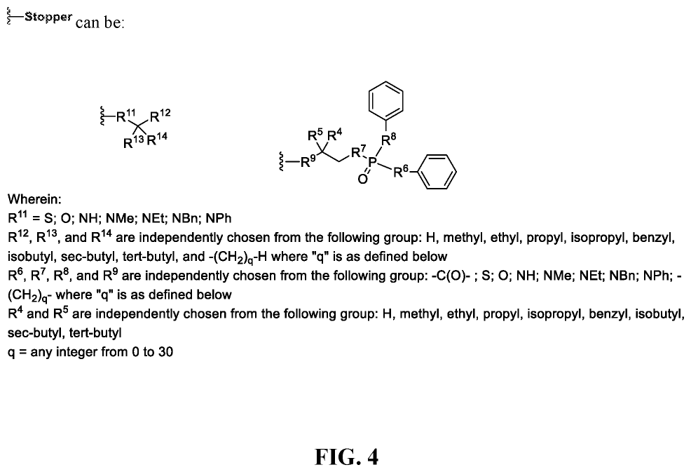

In another embodiment, an anchor can include a linker constituent (Linker) such as in addition to a GAP constituent and/or AA(Gap) constituent, such as with respect to anchors including H-AA(Gap)-OH, H-[AA¹]_{α}-[AA(Gap)]_{β}-[AA²]_{γ}-Stopper, H-Linker-[AA¹]_{α}-[AA(Gap)]_{β}-[AA²]_{γ}-Stopper, and/or H-Linker-{[AA1]_{α}-[AA(Gap)]_{β}-[AA2]_{γ}-Stopper}_{δ}. In one embodiment, a linker constituent can include linkers known in the in the art with respect to peptide synthesis. In another embodiment, and as illustrated in FIG. 3, a linker constituent

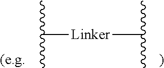

can include:

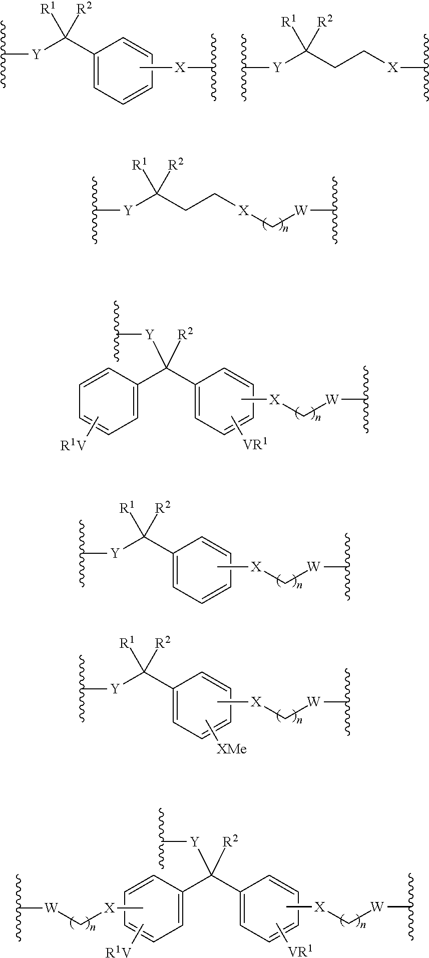

wherein Y can be S, NH, NMe, NEt, NBn, NPh, or O; V can be S, NH, NMe, NEt, NBn, NPh, O, —C(O)—, —(CH₂)ₐ₁—; W can be S, NH, NMe, NEt, NBn, NPh, O, —C(O)—, —(CH₂)ₐ₂—; X can be S, NH, NMe, NEt, NBn, NPh, O, —C(O)—, —(CH₂)ₐ₃—; R¹ can be —(CH₂)_{b}—H, —CCl₃, —CF₃, phenyl, isopropyl, tert-butyl, chlorophenyl, dichlorophenyl, methoxyphenyl, or dimethoxyphenyl; R² can be —(CH₂)_{c}—H, —CCl3, —CF3, phenyl, isopropyl, tert-butyl, chlorophenyl, dichlorophenyl, methoxyphenyl, or dimethoxyphenyl; and a, b, c, and n can be separate values that can be any integer from 0 up to and including 30. In one embodiment, the “H” (hydrogen) in “H-Linker” (as used in, e.g., H-Linker-[AA¹]_{α}-[AA(Gap)]_{β}-[AA²]_{γ}-Stopper and/or H-Linker-{ [AA1]_{α}-[AA(Gap)]_{β}-[AA2]_{γ}-Stopper}_{δ}) is attached to the “Y” variable of the Linker (such attachment notated above with a squiggly line).

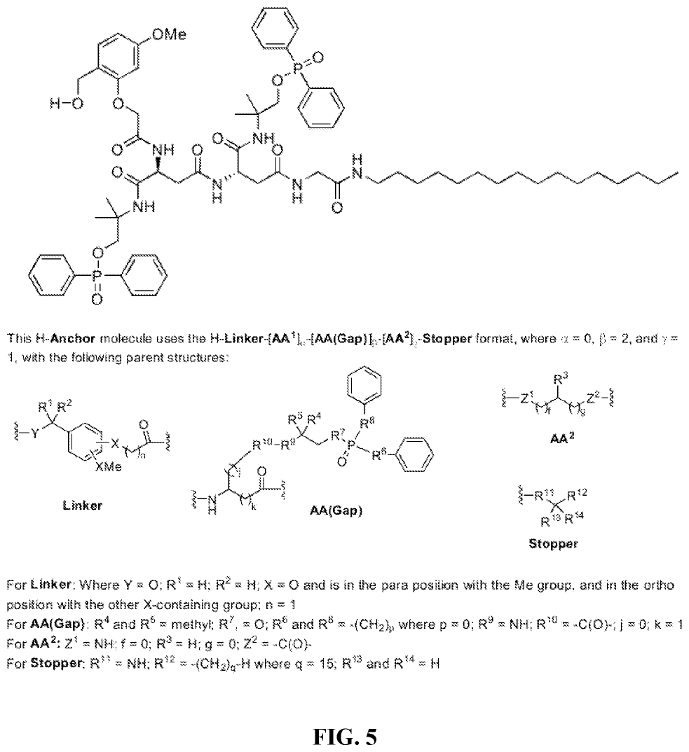

In another embodiment, an anchor can include a stopper constituent (Stopper) such as in addition to a GAP constituent and/or AA(Gap) constituent and/or linker constituent, such as with respect to anchors including H-AA(Gap)-OH, H-[AA¹]_{α}-[AA(Gap)]_{β}-[AA²]_{γ}-Stopper, H-Linker-[AA¹]_{α}-[AA(Gap)]_{β}-[AA²]_{γ}-Stopper, and/or H-Linker-{[AA1]_{α}-[AA(Gap)]_{β}-[AA2]_{γ}-Stopper}_{δ}. In one embodiment, and as illustrated in FIG. 4, a stopper constituent

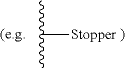

can include:

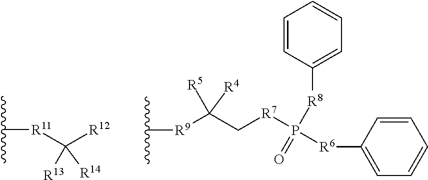

wherein R¹¹ can be S, O, NH, NMe, Net, NBn, or NPh; R¹², R¹³, and R¹⁴ can be any of H, methyl, ethyl, propyl, isopropyl, benzyl, isobutyl, sec-butyl, tert-butyl, and —(CH₂)_{q}—H; R⁶, R⁷, R⁸, and R⁹ can be any of —C(O)—, S, O, NH, NMe, Net, NBn, NPh, or —(CH2)q-; R⁴ and R⁵ can be any of H, methyl, ethyl, propyl, isopropyl, benzyl, isobutyl, sec-butyl, or tert-butyl; and q can be any integer from 0 up to and including 30. R⁴, R⁵, R⁶, R⁷, R⁸, and R⁹ can all be different from each other, and the value of q can be different for different R groups.

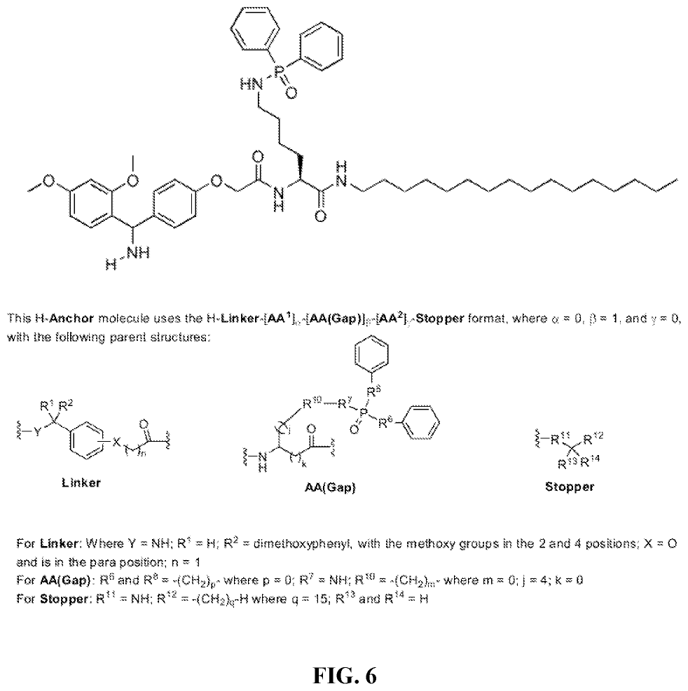

In another embodiment, the present disclosure can include compositions, such as compositions that can be useful as anchor molecules in accordance with the principles of the present disclosure. For example, and as illustrated in FIG. 5, an anchor molecule can include:

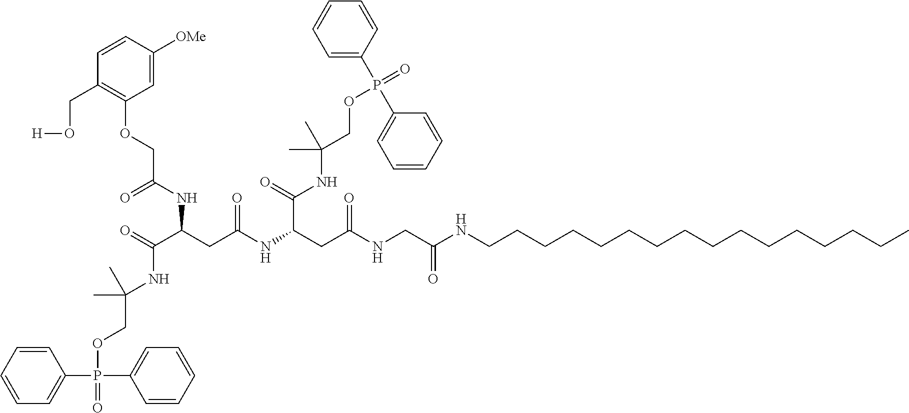 [SMILES: CCCCCCCCCCCCCCCCNC(=O)CNC(=O)C[C@H](NC(=O)C[C@H](NC(=O)COc1cc(OC)ccc1CO)C(=O)NC(C)(C)COP(=O)(c1ccccc1)c1ccccc1)C(=O)NC(C)(C)COP(=O)(c1ccccc1)c1ccccc1]

In this example, the anchor can follow the H-Linker-[AA¹]_{α}-[AA(Gap)]_{β}-[AA²]_{γ}-Stopper format, wherein a can be 0, β can be 2, and γ can be 1. In one embodiment each constituent can be defined as follows:

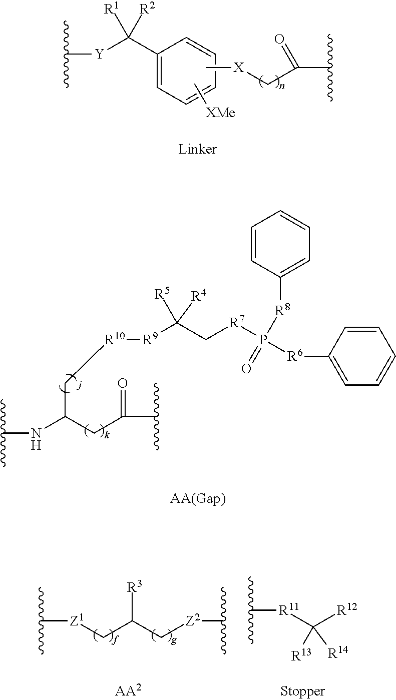

For Linker: Where Y═O; R¹═H; R²═H; X═O and is in the para position with the Me group, and in the ortho position with the other X-containing group; n=1
For AA(Gap): R⁴ and R⁵=methyl; R⁷, ═O; R⁶ and R⁸═—(CH₂)ₚ where p=0; R⁹═NH; R¹⁰═C(O)—; j=0; k=1
For AA²: Z¹═NH; f=0; R³═H; g=0; Z²═—C(O)—
For Stopper: R¹¹═NH; R¹²═—(CH₂)_{q}—H where q=15; R¹³ and R¹⁴═H

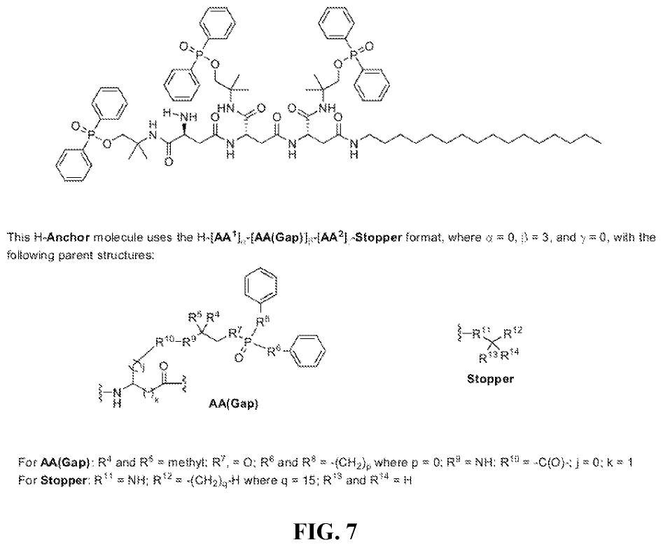

In another embodiment, and as shown in FIG. 6, an anchor molecule can include:

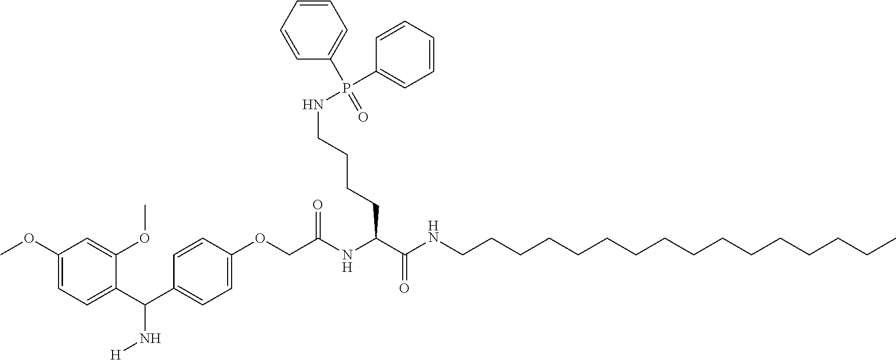 [SMILES: CCCCCCCCCCCCCCCCNC(=O)[C@H](CCCCNP(=O)(c1ccccc1)c1ccccc1)NC(=O)COc1ccc(C(N)c2ccc(OC)cc2OC)cc1]

In this example, the anchor can follow the H-Linker-[AA¹]_{α}-[AA(Gap)]_{β}-[AA²]_{γ}-Stopper format, wherein α can be 0, β can be 1, and γ can be 0. In one embodiment each constituent can be defined as follows:

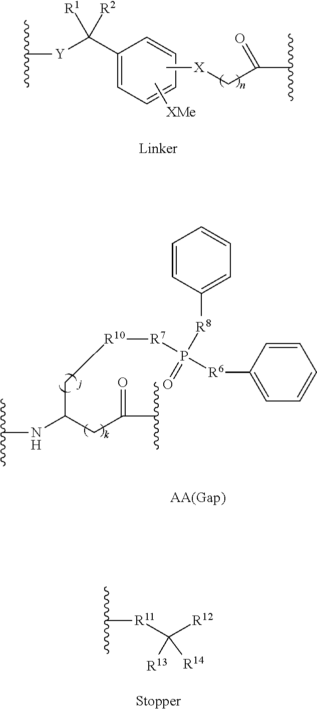

For Linker: Where Y═NH; R¹═H: R²=dimethoxyphenyl, with the methoxy groups in the 2 and 4 positions; X═O and is in the para position; n=1
For AA(Gap): R⁶ and R⁸═—(CH₂)ₚ— where p=0; R⁷═NH; R¹⁰═—(CH₂)ₘ— where m=0; j=4; k=0
For Stopper: R¹¹═NH; R¹²═—(CH₂)_{q}—H where q=15; R¹³ and R¹⁴═H

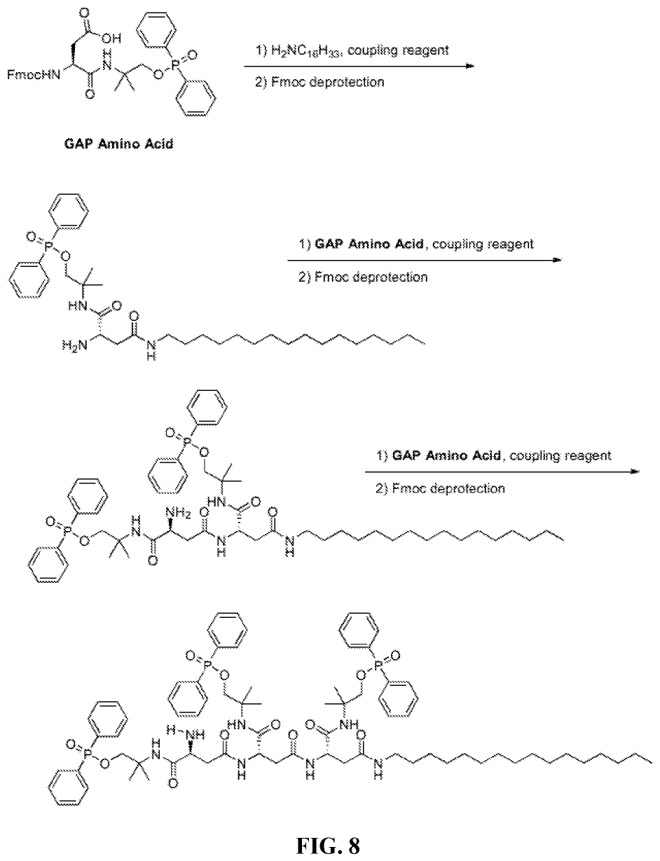

In another embodiment, and as shown in FIG. 7, an anchor molecule can include:

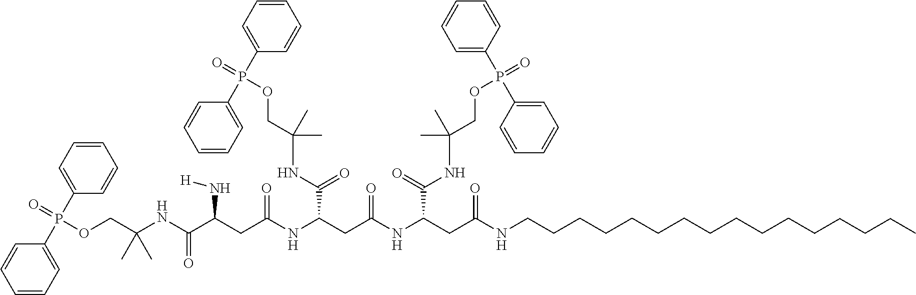 [SMILES: CCCCCCCCCCCCCCCCNC(=O)C[C@H](NC(=O)C[C@H](NC(=O)C[C@H](N)C(=O)NC(C)(C)COP(=O)(c1ccccc1)c1ccccc1)C(=O)NC(C)(C)COP(=O)(c1ccccc1)c1ccccc1)C(=O)NC(C)(C)COP(=O)(c1ccccc1)c1ccccc1]

In this example, the anchor can follow the H-[AA¹]_{α}-[AA(Gap)]_{β}-[AA²]_{γ}-Stopper format, wherein α can be 0, β can be 3, and γ can be 0. In one embodiment each constituent can be defined as follows:

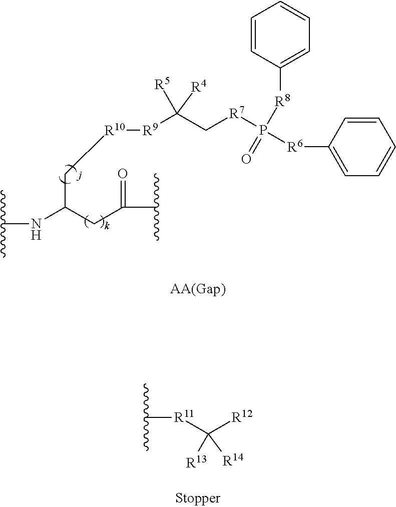

For AA(Gap): R⁴ and R⁵=methyl; R⁷, ═O; R⁶ and R⁸═—(CH₂)ₚ where p=0; R⁹═NH; R¹⁰═—C(O)—; j=0; k=1
For Stopper: R¹¹═NH; R¹²═—(CH₂)_{q}—H where q=15; R¹³ and R¹⁴═H

In another embodiment, and as shown in FIG. 9, an anchor molecule can include:

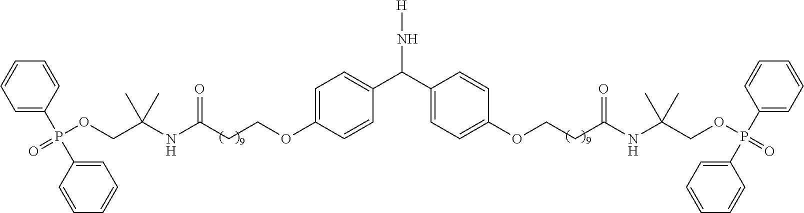

In this example, the anchor can follow the H-Linker-{[AA¹]_{α}-[AA(Gap)]_{β}-[AA²]_{γ}-Stopper}_{δ} format, wherein α can be 0, β can be 0, γ can be 0, and δ can be 2. In one embodiment, each constituent can be defined as follows:

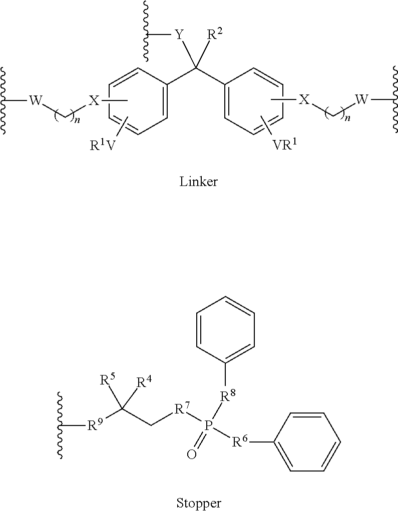

For Linker: Y═NH; V═—(CH₂)_{α}— where a=0; X═O; W═—C(O)—; R¹═(CH₂)_{b}—H where b=0; R²═—(CH₂)_{c}—H where c=0; n=10
For Stopper: R⁹═NH; R⁵ and R⁴=methyl; R⁶ and R⁸═—(CH₂)_{q}—where q=0; R⁷═O

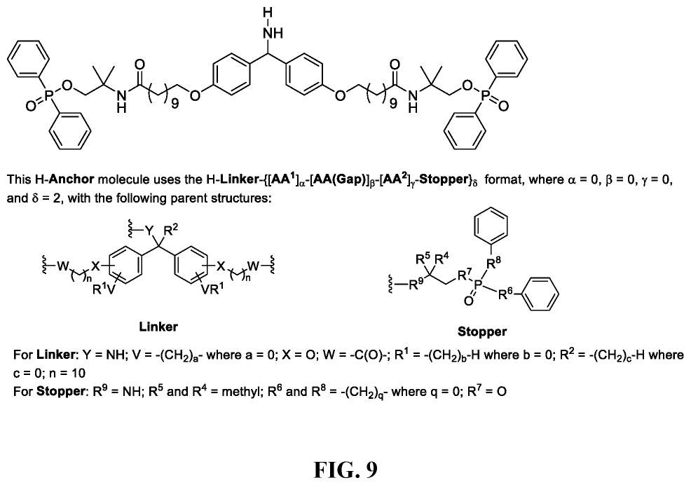

In another embodiment, the present disclosure can include a method of synthesizing an anchor (in some embodiments, an anchor peptide). In one embodiment, the method can be modular system, such that an initial starting material (e.g., an AA(Gap) constituent) can be made in bulk, and then incorporated into an anchor to varying degrees such that the anchor can have different properties depending on the degree of incorporation of the starting material. For example, with respect to the synthesis of the anchor depicted in FIG. 7, an AA(Gap) constituent can be a GAP amino acid (e.g., an amino acid with a protecting group, preferably a GAP protecting group, attached thereto) and can further act as a modular starting material. For example, and as depicted in FIG. 8, a synthesis method can begin with:

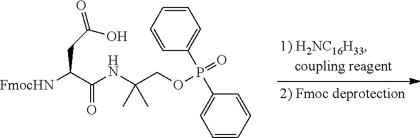

In one embodiment, this step can be considered an attachment of an AA(Gap) constituent to a stopper constituent (e.g. H₂NC₁₆H₃₃). The product of the above reaction can be:

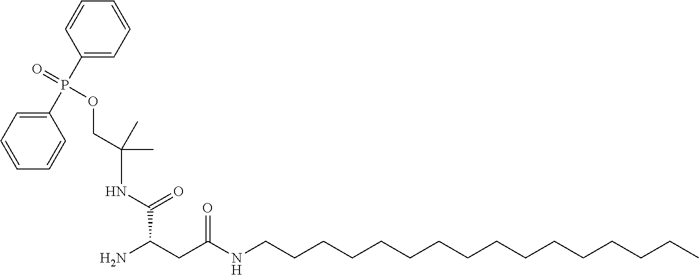 [SMILES: CCCCCCCCCCCCCCCCNC(=O)C[C@H](N)C(=O)NC(C)(C)COP(=O)(c1ccccc1)c1ccccc1]

In one embodiment, the above molecule can then be coupled to a first amino acid of a peptide, or alternatively, a linker constituent can be further incorporated before attachment to a first amino acid. In another embodiment, the present disclosure can further include the addition of one or more further AA(Gap) constituents to the above molecule. For example, for the synthesis of a longer peptide sequence (e.g., between 25 amino acids and 100 amino acids), it can be advantageous to include at least one other AA(Gap) constituent in the anchor, such that the solubility of the target peptide can be further controlled as the sequence lengthens. In one example, the above molecule can be reacted with another AA(Gap) constituent (e.g., the same or different AA(Gap) constituent as used previously) to achieve:

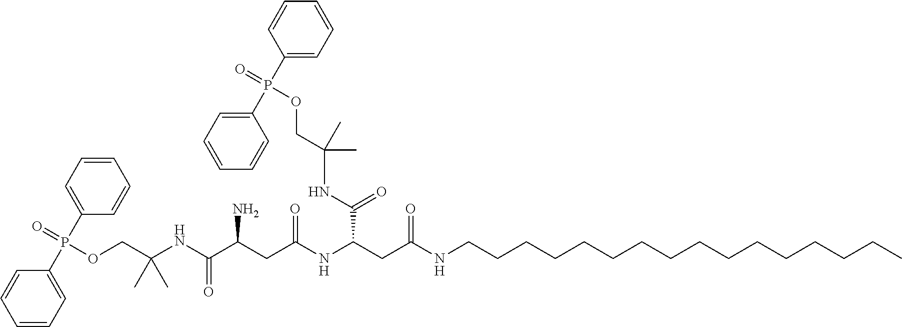 [SMILES: CCCCCCCCCCCCCCCCNC(=O)C[C@H](NC(=O)C[C@H](N)C(=O)NC(C)(C)COP(=O)(c1ccccc1)c1ccccc1)C(=O)NC(C)(C)COP(=O)(c1ccccc1)c1ccccc1]

In one embodiment, the above molecule can then be coupled to a first amino acid of a peptide, or alternatively, a linker constituent can be further incorporated before attachment to a first amino acid. In another embodiment, the present disclosure can further include the addition of one or more further AA(Gap) constituents to the above molecule. For example, the above molecule can be coupled to another AA(Gap) constituent to yield:

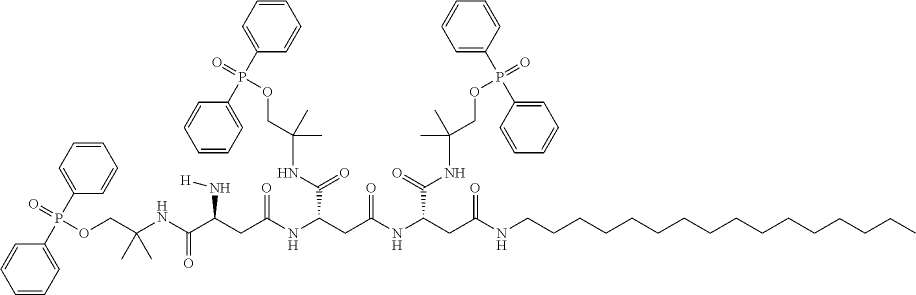 [SMILES: CCCCCCCCCCCCCCCCNC(=O)C[C@H](NC(=O)C[C@H](NC(=O)C[C@H](N)C(=O)NC(C)(C)COP(=O)(c1ccccc1)c1ccccc1)C(=O)NC(C)(C)COP(=O)(c1ccccc1)c1ccccc1)C(=O)NC(C)(C)COP(=O)(c1ccccc1)c1ccccc1]

In one embodiment, the above molecule can then be coupled to a first amino acid of a peptide, or alternatively, a linker constituent can be further incorporated before attachment to a first amino acid. In another embodiment, the present disclosure can further include the addition of one or more further AA(Gap) constituents to the above molecule.

The method exemplified in FIG. 8 can be considered a modular system, in that one or more units (e.g. an AA(Gap) constituent) can be added multiple times to achieve greater and greater solubility control, as needed. Such modularity is advantageous in the art because it enables the large-scale synthesis and stockpiling of starting materials that can then be converted into customized anchors as needed. It will be understood by those having skill in the art that multiple different amino acids can be used in this method to lend different characteristics to the anchor. It will be further understood that the stopper constituent can be modified to lend desired characteristics, such as increased hydrophobicity, increased hydrophilicity, increased aromaticity, and/or any other desired quality. Coupling and deprotection reactions can occur in one or more solvents, such as, e.g., dichloromethane, DMF, ethyl acetate, tetrahydrofuran, and/or 2-methyltetrahydrofuran.

In one embodiment, the GAP, linker, amino acid, and stopper constituents can be considered as separate chemical entities capable of lending different useful properties to a given GAP anchor. For example, the GAP constituent can include a GAP molecule coupled to an amino acid that includes a polar bond (e.g. phosphine oxide) and aromatic constituents, which ultimately give the anchor a polar-organic quality with potential for pi-pi stacking, which can assist the anchor (and attached target molecule) in selectively precipitating from non-polar solvents; in another embodiment, this can assist in the anchor in excluding itself from aqueous solvents and non-polar solvents. In one embodiment, in the same anchor, a linker constituent can provide the anchor with the ability to be selectively removed from a desired substrate while additionally contributing desired solubility properties to the anchor. For example, a linker constituent can be specifically designed to form a non-labile bond at one location, and additionally form a labile bond at another location. This orthogonality can ultimately be translated to the anchor; the GAP constituent and linker constituent together can, in this manner, provide a GAP anchor with polar-organic qualities lending itself to group-assisted purification chemistry, that additionally can be cleaved under conditions the linker constituent itself would otherwise be amenable to. In another embodiment, in the same (or different) anchor, a stopper constituent can provide further desired characteristics to the anchor. For example, a stopper can be highly organic or non-polar, increasing the overall solubility of the anchor in non-polar solvents; in another example, the stopper can be highly polar, such that the anchor can also be more polar and therefore less soluble in non-polar, organic solvents.

In accordance with the principles of the present disclosure, an anchor can thereby be specially formulated to assist in the synthesis of a given molecule with particular characteristics. For example, and as discussed further herein, a desired peptide can be synthesized with assistance from an anchor. A GAP constituent (including an AA(Gap) constituent) of the anchor can have qualities discussed herein that help render the anchor (and attached peptide) soluble in organic solvents; the GAP constituent can also be amenable to pi-pi stacking to assist the peptide in selective precipitation, such as via drop precipitation. These characteristics can be further accentuated in an anchor by including more than one AA(Gap) constituent in one anchor molecule. For example, two AA(Gap) constituents can be coupled together via the termini of the amino acids therein, such as via well-known coupling reactions, to form a protected peptide. In one embodiment, this GAP-protected peptide can be incorporated into an anchor molecule for peptide synthesis. In another embodiment, a stopper constituent can also be included in the anchor—in one embodiment, a stopper constituent can include an aliphatic amine and/or alcohol and/or carboxylic acid capable of coupling to a terminus of an amino acid. In another embodiment, a linker constituent can also be included, and lend a free moiety capable of participating in a reaction, such as a coupling reaction. Preferably, the bond formed by the free moiety of the linker constituent can be labile to, for example, a TFA-deprotection known in the art like those discussed herein.

It should be understood that the constituents addressed herein are described separately to better communicate the different characteristics that each constituent can contribute to a given anchor molecule, and the separate descriptions of the constituents herein should not be considered as limiting. It should be further understood that any of the constituents discussed herein can be considered protecting groups. For example, GAP constituents, AA(Gap) constituents, linker constituents, and stopper constituents can attach to a given molecule and protect that area of the molecule from unwanted side reactions. The anchors discussed herein can further be considered protecting groups in-and-of themselves. In another example, anchors discussed herein can be attached to a C-terminus of an amino acid, such as via a nucleophilic moiety (e.g., NH, OH, etc.) that can attach a carbonyl carbon.

1. A chemical composition comprising a GAP constituent, wherein the GAP constituent is selected from the group consisting of: 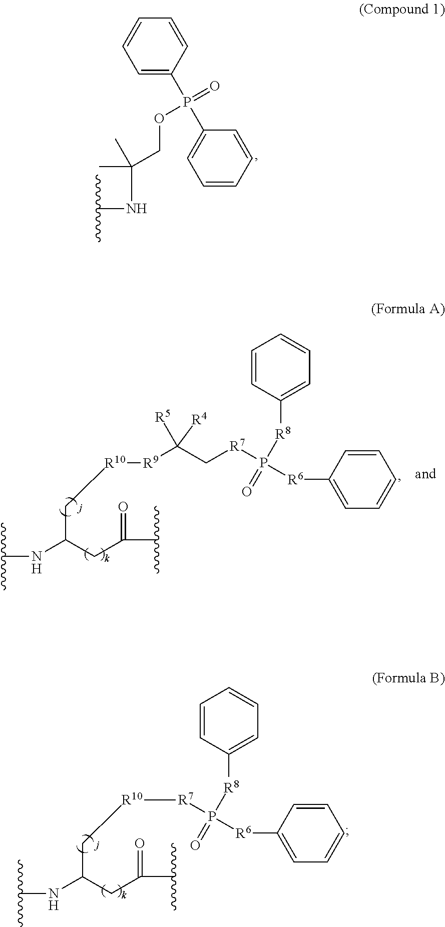
wherein:
R⁴ is selected from the group consisting of: H, methyl, ethyl, propyl, isopropyl, benzyl, isobutyl, sec-butyl, and tert-butyl;
R⁵ is selected from the group consisting of: H, methyl, ethyl, propyl, isopropyl, benzyl, isobutyl, sec-butyl, and tert-butyl;
R⁶ is selected from the group consisting of: —C(O)—, S, O, NH, NMe, Net, NBn, NPh, and —(CH₂)ₚ₁—, wherein “p1” is an integer selected from the range of 0 up to and including 30;
R⁷ is selected from the group consisting of: —C(O)—, S, O, NH, NMe, Net, NBn, NPh, and —(CH₂)ₚ₂—, wherein “p2” is an integer selected from the range of 0 up to and including 30;
R⁸ is selected from the group consisting of: —C(O)—, S, O, NH, NMe, Net, NBn, NPh, and —(CH₂)ₚ₃—, wherein “p3” is an integer selected from the range of 0 up to and including 30;
R⁹ is selected from the group consisting of: —C(O)—, S, O, NH, NMe, Net, NBn, NPh, and —(CH₂)ₚ₄—, wherein “p4” is an integer selected from the range of 0 up to and including 30;
R¹⁰ is selected from the group consisting of: —C(O)—, —(CH₂)ₘ—, and NH, wherein “m” is an integer selected from the range of 0 up to and including 30;
“j” is an integer selected from the range of 0 up to and including 30; and
“k” is an integer selected from the range of 0 up to and including 30.

2. The chemical composition of claim 1, further comprising a linker constituent, wherein the linker constituent is selected from the group consisting of: 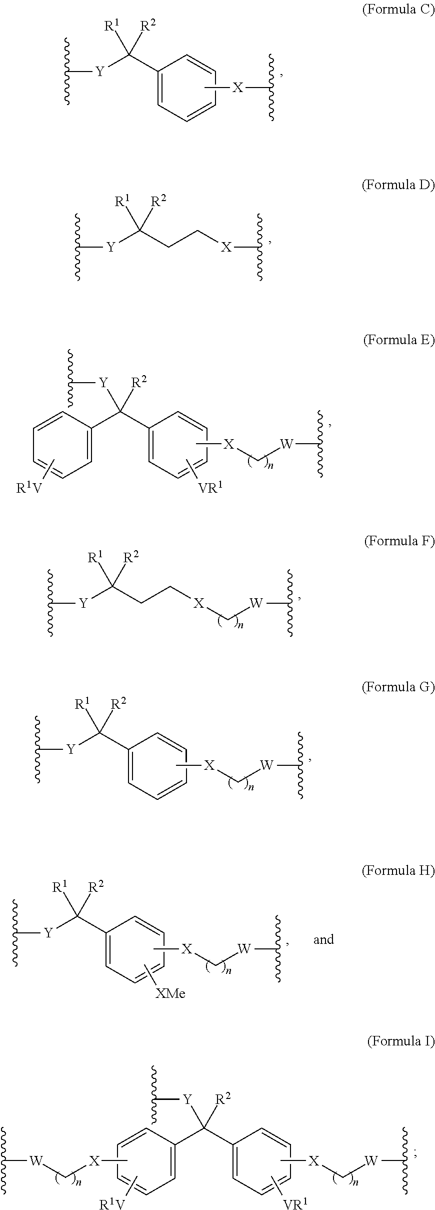
wherein:
Y is selected from the group consisting of: S, NH, NMe, NEt, NBn, NPh, and O;
V is selected from the group consisting of: S, NH, NMe, NEt, NBn, NPh, O, —C(O)—, —(CH₂)ₐ₁—, wherein “a1” is an integer selected from the range of 0 up to and including 30;
W is selected from the group consisting of: S, NH, NMe, NEt, NBn, NPh, O, —C(O)—, —(CH₂)ₐ₂—, wherein “a2” is an integer selected from the range of 0 up to and including 30;
X is selected from the group consisting of: S, NH, NMe, NEt, NBn, NPh, O, —C(O)—, —(CH₂)ₐ₃—, wherein “a3” is an integer selected from the range of 0 up to and including 30;
R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30;
R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and
“n” is an integer selected from the range of 0 up to and including 30.

3. The chemical composition of claim 1, further comprising a stopper constituent, wherein the stopper constituent is selected from the group consisting of: 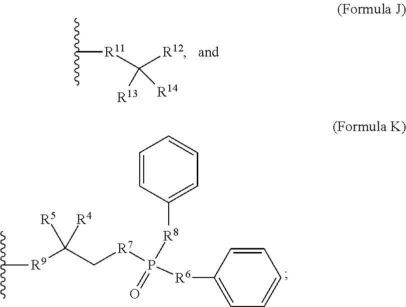
wherein:
R⁴ is selected from the group consisting of: H, methyl, ethyl, propyl, isopropyl, benzyl, isobutyl, sec-butyl, and tert-butyl;
R⁵ is selected from the group consisting of: H, methyl, ethyl, propyl, isopropyl, benzyl, isobutyl, sec-butyl, and tert-butyl;
R⁶ is selected from the group consisting of: —C(O)—, S, O, NH, NMe, Net, NBn, NPh, and —(CH2)_{q1}-, wherein “q1” is an integer selected from the range of 0 up to and including 30;
R⁷ is selected from the group consisting of: —C(O)—, S, O, NH, NMe, Net, NBn, NPh, and —(CH2)_{q2}-, wherein “q2” is an integer selected from the range of 0 up to and including 30;
R⁸ is selected from the group consisting of: —C(O)—, S, O, NH, NMe, Net, NBn, NPh, and —(CH2)_{q3}-, wherein “q3” is an integer selected from the range of 0 up to and including 30;
R⁹ is selected from the group consisting of: —C(O)—, S, O, NH, NMe, Net, NBn, NPh, and —(CH2)_{q4}-, wherein “q4” is an integer selected from the range of 0 up to and including 30;
R¹¹ is selected from the group consisting of: S, O, NH, NMe, Net, NBn, and NPh;
R¹² is selected from the group consisting of: H, methyl, ethyl, propyl, isopropyl, benzyl, isobutyl, sec-butyl, tert-butyl, and —(CH₂)_{q5}—H, wherein “q5” is an integer selected from the range of 0 up to and including 30;
R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and
R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30.

4. The chemical composition of claim 1, further comprising a linker constituent, a stopper constituent, and a first amino acid constituent, wherein the GAP constituent is 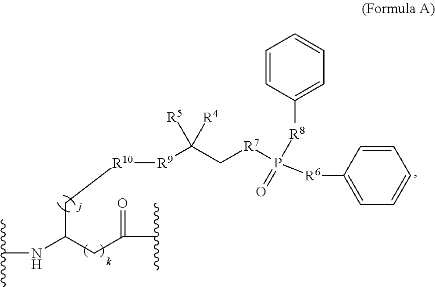
wherein:
R⁴ is selected from the group consisting of: H, methyl, ethyl, propyl, isopropyl, benzyl, isobutyl, sec-butyl, and tert-butyl;
R⁵ is selected from the group consisting of: H, methyl, ethyl, propyl, isopropyl, benzyl, isobutyl, sec-butyl, and tert-butyl;
R⁶ is selected from the group consisting of: —C(O)—, S, O, NH, NMe, Net, NBn, NPh, and —(CH₂)ₚ₁—, wherein “p1” is an integer selected from the range of 0 up to and including 30;
R⁷ is selected from the group consisting of: —C(O)—, S, O, NH, NMe, Net, NBn, NPh, and —(CH₂)ₚ₂—, wherein “p2” is an integer selected from the range of 0 up to and including 30;
R⁸ is selected from the group consisting of: —C(O)—, S, O, NH, NMe, Net, NBn, NPh, and —(CH₂)ₚ₃—, wherein “p3” is an integer selected from the range of 0 up to and including 30;
R⁹ is selected from the group consisting of: —C(O)—, S, O, NH, NMe, Net, NBn, NPh, and —(CH₂)ₚ₄—, wherein “p4” is an integer selected from the range of 0 up to and including 30;
R¹⁰ is selected from the group consisting of: —C(O)—, —(CH₂)ₘ—, and NH, wherein “m” is an integer selected from the range of 0 up to and including 30;
“j” is an integer selected from the range of 0 up to and including 30; and
“k” is an integer selected from the range of 0 up to and including 30;
the linker constituent is 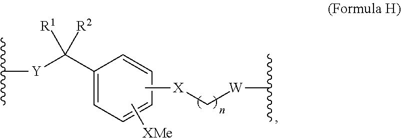
wherein:
Y is selected from the group consisting of: S, NH, NMe, NEt, NBn, NPh, and O;
W is —C(O)—;
X is selected from the group consisting of: S, NH, NMe, NEt, NBn, NPh, O, —C(O)—, —(CH₂)ₐ₃—, wherein “a3” is an integer selected from the range of 0 up to and including 30;
R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30;
R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and
“n” is an integer selected from the range of 0 up to and including 30;
the first amino acid constituent is 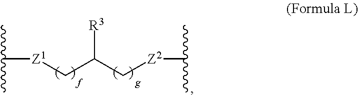
wherein Z¹ is selected from the group consisting of: S, O, NH, NMe, Net, NBn, NPh, —C(O)—, and —(CH₂)_{d}—, wherein “d” is an integer selected from the range of 0 up to and including 30;
Z² is selected from the group consisting of: S, O NH, NMe, Net, NBn, NPh, —C(O)—, and —(CH₂)ₑ—, wherein “e” is an integer selected from the range of 0 up to and including 30;
“f” is an integer selected from the range of 0 up to and including 30;
“g” is an integer selected from the range of 0 up to and including 30;
R³ is selected from the group consisting of: H, methyl, ethyl, propyl, isopropyl, benzyl, isobutyl, sec-butyl, tert-butyl, 2-(methylmercapto)ethyl, 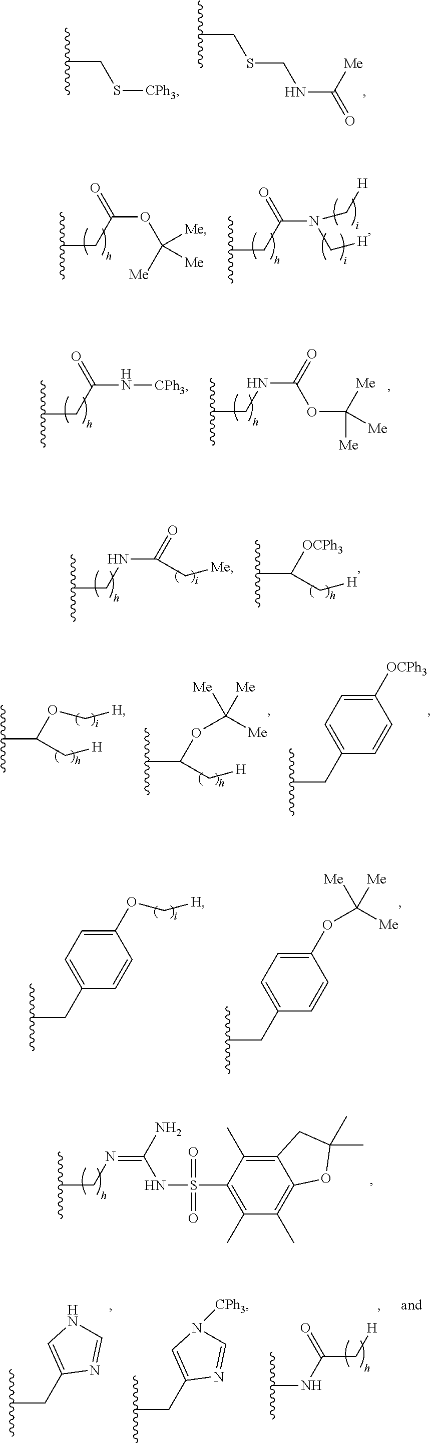 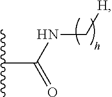
wherein “h” is an integer selected from the range of 0 up to and including 30; and
the stopper constituent is 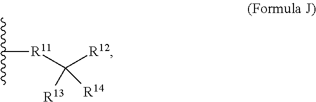
wherein:
R¹¹ is selected from the group consisting of: S, O, NH, NMe, Net, NBn, and NPh;
R¹² is selected from the group consisting of: H, methyl, ethyl, propyl, isopropyl, benzyl, isobutyl, sec-butyl, tert-butyl, and —(CH₂)_{q5}—H, wherein “q5” is an integer selected from the range of 0 up to and including 30;
R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and
R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30.

5. The chemical composition of claim 4, comprising:

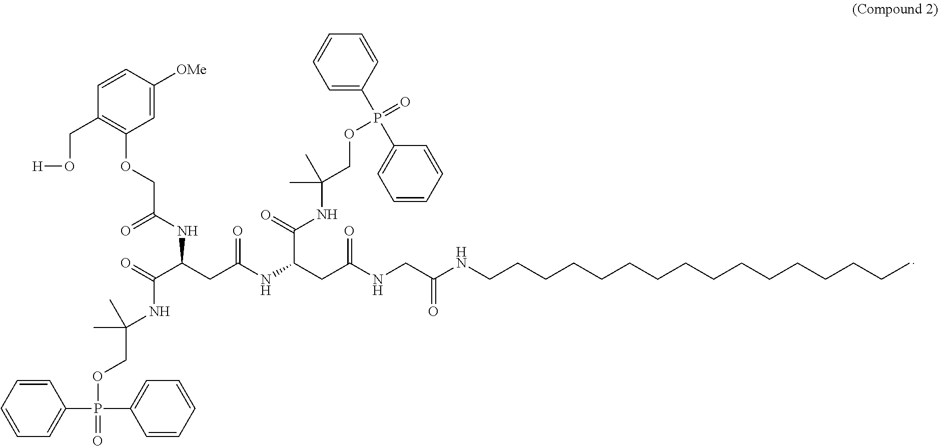 [SMILES: CCCCCCCCCCCCCCCCNC(=O)CNC(=O)C[C@H](NC(=O)C[C@H](NC(=O)COc1cc(OC)ccc1CO)C(=O)NC(C)(C)COP(=O)(c1ccccc1)c1ccccc1)C(=O)NC(C)(C)COP(=O)(c1ccccc1)c1ccccc1]

6. The chemical composition of claim 1, further comprising a linker constituent and a stopper constituent, wherein the GAP constituent is 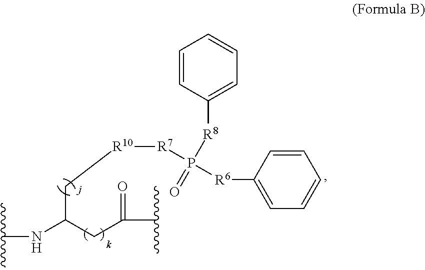
wherein:
R⁶ is selected from the group consisting of: —C(O)—, S, O, NH, NMe, Net, NBn, NPh, and —(CH₂)ₚ₁—, wherein “p1” is an integer selected from the range of 0 up to and including 30;
R⁷ is selected from the group consisting of: —C(O)—, S, O, NH, NMe, Net, NBn, NPh, and —(CH₂)ₚ₂—, wherein “p2” is an integer selected from the range of 0 up to and including 30;
R⁸ is selected from the group consisting of: - C(O)-, S, O, NH, NMe, Net, NBn, NPh, and —(CH₂)ₚ₃—, wherein “p3” is an integer selected from the range of 0 up to and including 30;
R¹⁰ is selected from the group consisting of: —C(O)—, —(CH₂)ₘ—, and NH, wherein “m” is an integer selected from the range of 0 up to and including 30;
“j” is an integer selected from the range of 0 up to and including 30; and
“k” is an integer selected from the range of 0 up to and including 30;
the linker constituent is 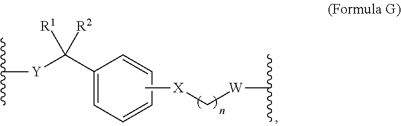
wherein:
Y is selected from the group consisting of: S, NH, NMe, NEt, NBn, NPh, and O;
W is —C(O)—;
X is selected from the group consisting of: S, NH, NMe, NEt, NBn, NPh, O, —C(O)—, —(CH₂)ₐ₃—, wherein “a3” is an integer selected from the range of 0 up to and including 30;
R¹ is selected from the group consisting of —(CH₂)_{b}—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30;
R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and
“n” is an integer selected from the range of 0 up to and including 30; and
the stopper constituent is 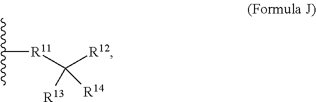
wherein:
R¹¹ is selected from the group consisting of: S, O, NH, NMe, Net, NBn, and NPh;
R¹² is selected from the group consisting of: H, methyl, ethyl, propyl, isopropyl, benzyl, isobutyl, sec-butyl, tert-butyl, and —(CH₂)_{q5}—H, wherein “q5” is an integer selected from the range of 0 up to and including 30;
R¹³ is selected from the group consisting of: H, methyl, ethyl, propyl, isopropyl, benzyl, isobutyl, sec-butyl, tert-butyl, and —(CH₂)_{q6}—H, wherein “q6” is an integer selected from the range of 0 up to and including 30; and
R¹⁴ is selected from the group consisting of: H, methyl, ethyl, propyl, isopropyl, benzyl, isobutyl, sec-butyl, tert-butyl, and —(CH₂)_{q7}—H, wherein “q7” is an integer selected from the range of 0 up to and including 30.

7. The chemical composition of claim 6, comprising:

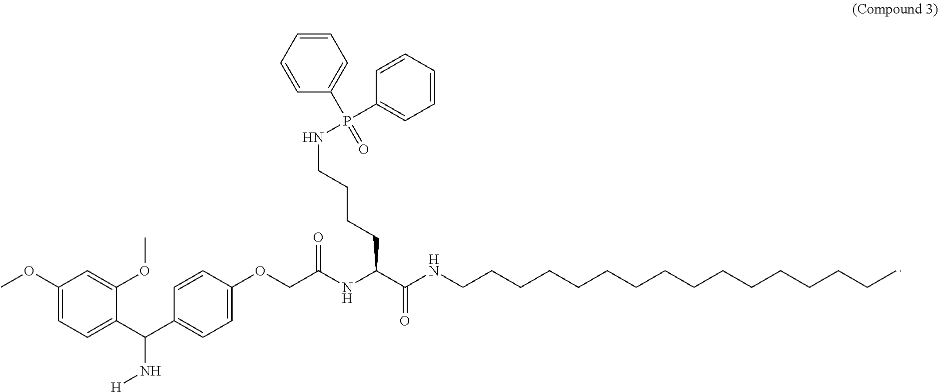 [SMILES: CCCCCCCCCCCCCCCCNC(=O)[C@H](CCCCNP(=O)(c1ccccc1)c1ccccc1)NC(=O)COc1ccc(C(N)c2ccc(OC)cc2OC)cc1]

8. The chemical composition of claim 1, further comprising a linker constituent and a stopper constituent, wherein the GAP constituent is 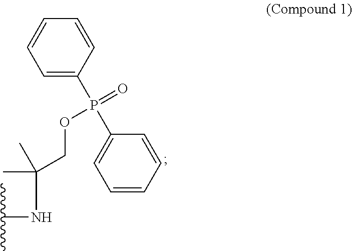 [SMILES: *NC(C)(C)COP(=O)(c1ccccc1)c1ccccc1]
the linker constituent is 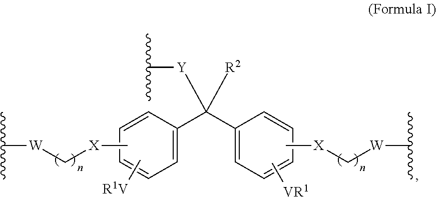
wherein:
Y is selected from the group consisting of: S, NH, NMe, NEt, NBn, NPh, and O;
V is selected from the group consisting of: S, NH, NMe, NEt, NBn, NPh, O, —C(O)—, —(CH₂)ₐ₁—, wherein “a1” is an integer selected from the range of 0 up to and including 30;
W is selected from the group consisting of: S, NH, NMe, NEt, NBn, NPh, O, —C(O)—, —(CH₂)ₐ₂—, wherein “a2” is an integer selected from the range of 0 up to and including 30;
X is selected from the group consisting of: S, NH, NMe, NEt, NBn, NPh, O, —C(O)—, —(CH₂)ₐ₃—, wherein “a3” is an integer selected from the range of 0 up to and including 30;
R¹ is selected from the group consisting of —(CH₂)₆—H, —CCl₃, —CF₃, phenyl, isopropyl, tert-butyl, chlorophenyl, dichlorophenyl, methoxyphenyl, and dimethoxyphenyl, wherein “b” is an integer selected from the range of 0 up to and including 30;
R² is selected from the group consisting of —(CH₂)_{c}—H, —CCl3, —CF3, phenyl, isopropyl, tert-butyl, chlorophenyl, dichlorophenyl, methoxyphenyl, and dimethoxyphenyl, wherein “c” is an integer selected from the range of 0 up to and including 30; and
“n” is an integer selected from the range of 0 up to and including 30; and
the stopper constituent is 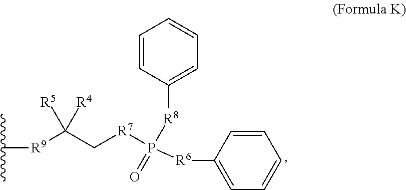
wherein:
R⁴ is selected from the group consisting of: H, methyl, ethyl, propyl, isopropyl, benzyl, isobutyl, sec-butyl, and tert-butyl;
R⁵ is selected from the group consisting of: H, methyl, ethyl, propyl, isopropyl, benzyl, isobutyl, sec-butyl, and tert-butyl;
R⁶ is selected from the group consisting of: —C(O)—, S, O, NH, NMe, Net, NBn, NPh, and —(CH2)_{q1}-, wherein “q1” is an integer selected from the range of 0 up to and including 30;
R⁷ is selected from the group consisting of: —C(O)—, S, O, NH, NMe, Net, NBn, NPh, and —(CH2)_{q2}-, wherein “q2” is an integer selected from the range of 0 up to and including 30;
R⁸ is selected from the group consisting of: —C(O)—, S, O, NH, NMe, Net, NBn, NPh, and —(CH2)_{q3}-, wherein “q3” is an integer selected from the range of 0 up to and including 30; and
R⁹ is selected from the group consisting of: —C(O)—, S, O, NH, NMe, Net, NBn, NPh, and —(CH2)_{q4}-, wherein “q4” is an integer selected from the range of 0 up to and including 30.

9. The chemical composition of claim 8, comprising

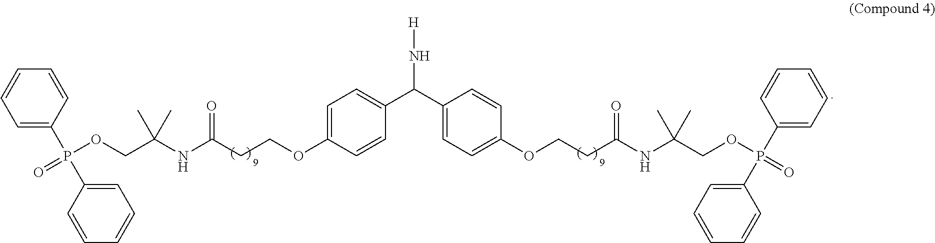

10. A method of solution-phase peptide synthesis, the method comprising the steps of:
attaching a first peptide to a first amino acid, wherein the first peptide is an anchor peptide;
coupling one or more additional amino acids to the first amino acid to form a second peptide; and
removing the first peptide from the second peptide.

11. The method of claim 10, wherein the first peptide includes a GAP constituent.

12. The method of claim 10, wherein the first peptide is formed by coupling a first protected amino acid to a second protected amino acid and attaching a linker to either the first or the second protected amino acid, wherein the first protected amino acid is formed by attaching a first protecting group to a first side chain of a first amino acid constituent, and the second protected amino acid is formed by coupling a second protecting group to a second side chain of a second amino acid constituent.

13. The method of claim 12, further comprising the steps of:
coupling the first amino acid constituent with the second amino acid constituent;
attaching the linker to a terminus of the first or second amino acid constituent;
attaching the linker to a third amino acid; and
coupling a fourth amino acid with the third amino acid.

14. A method of peptide synthesis, comprising the steps of:
coupling an anchor to a first amino acid; and
coupling a second amino acid with the first amino acid,
wherein the anchor includes a compound selected from the group consisting of:

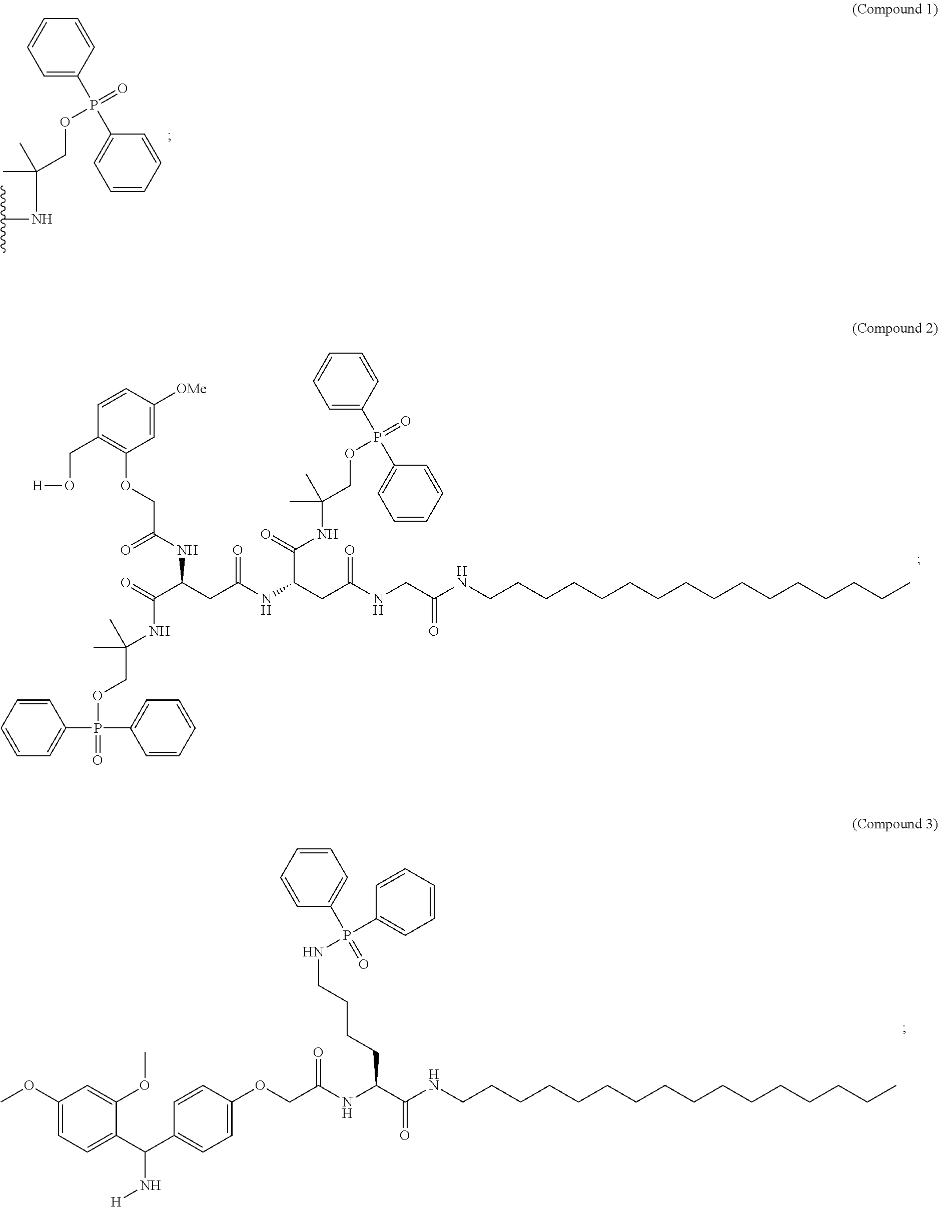

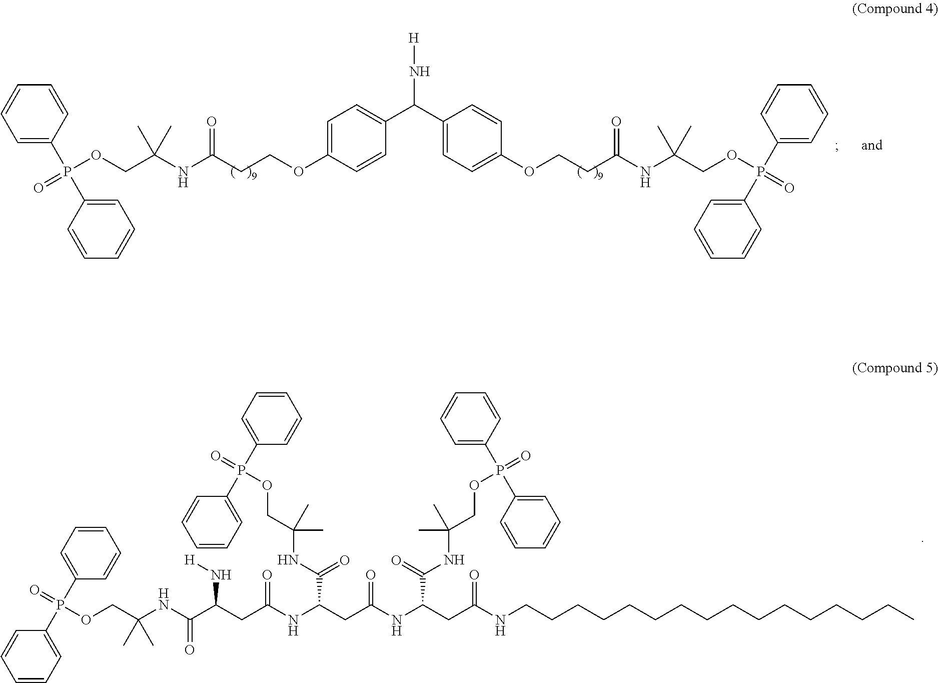

15. The method of claim 14, wherein the anchor is coupled to a C-terminus of the first amino acid.

16. The method of claim 14, wherein the anchor is coupled to a side chain of the first amino acid.

17. The method of claim 15, wherein the compound is 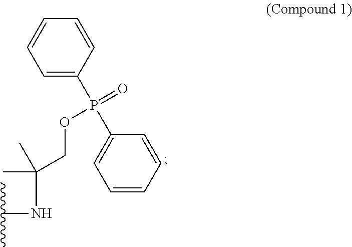 [SMILES: *NC(C)(C)COP(=O)(c1ccccc1)c1ccccc1]
and wherein the anchor is coupled to the C-terminus of the first amino acid via a linker constituent.

18. The method of claim 17, wherein the anchor further includes a stopper constituent.

19. The method of claim 14, further comprising the step of removing the anchor from the first amino acid.

20. The method claim 14, wherein the coupling of the second amino acid with the first amino acid occurs in 2-methyltetrahydrofuran.
